SEQUENCE LISTING (23 sequences)

SEQ ID NO: 1 - protein, 11 aa, Homo sapiens
RASQSVSSAVA

SEQ ID NO: 2 - protein, 7 aa, Homo sapiens
SASSLYS

SEQ ID NO: 3 - protein, 9 aa, Homo sapiens
QQSSTDPIT

SEQ ID NO: 4 - protein, 9 aa, Homo sapiens
FTVSSSSIH

SEQ ID NO: 5 - protein, 17 aa, Homo sapiens
YISSSSGYTYYADSVKG

SEQ ID NO: 6 - protein, 15 aa, Homo sapiens
YWSYPSWWPYRGMDY

SEQ ID NO: 7 - protein, 124 aa, Homo sapiens
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSS

SEQ ID NO: 8 - protein, 108 aa, Homo sapiens
DIQMTQSPSSLSASVGDRVTITCRASQSVSSAVAWYQQKPGKAPKLLIYSASSLYSGVPSRFSGSRSGTDFTLTISSLQPEDFATYYCQQSSTDPITFGQGTKVEIKR

SEQ ID NO: 9 - protein, 20 aa, Homo sapiens
MYRMQLLSCIALSLALVTNS

SEQ ID NO: 10 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 14 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 15 - protein, 214 aa, Homo sapiens
DIQMTQSPSSLSASVGDRVTITCRASQSVSSAVAWYQQKPGKAPKLLIYSASSLYSGVPSRFSGSRSGTDFTLTISSLQPEDFATYYCQQSSTDPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 454 aa, Homo sapiens
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 454 aa, Homo sapiens
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 451 aa, Homo sapiens
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 19 - protein, 451 aa, Homo sapiens
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 20 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 22 - protein, 451 aa, Homo sapiens
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 23 - protein, 451 aa, Homo sapiens
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

(19) United States
(12) Patent Application Publication
(10) Pub. No.: US 2022/0178930 A1
(43) Pub. Date: Jun. 9, 2022

(54) ANTI-GALECTIN-9 ANTIBODIES AND USES THEREOF

(71) Applicants: New York University, New York, NY (US); PureTech LYT, Inc., Boston, MA (US)

(72) Inventors: Shohei KOIDE, New York, NY (US); George MILLER, New York, NY (US); Akiko KOIDE, New York, NY (US); Linxiao CHEN, New York, NY (US); Aleksandra FILIPOVIC, London (GB); Eric ELENKO, Boston, MA (US); Joseph BOLEN, Boston, MA (US)

(21) Appl. No.: 17/607,879

(22) PCT Filed: May 1, 2020
(86) PCT No.: PCT/US2020/031184

§ 371 Date: Oct. 30, 2021

Related U.S. Application Data
(60) Provisional application No. 62/841,732, filed on May 1, 2019.

(51) Int. Cl. G01N 33/574; C07K 16/28

(52) U.S. Cl. CPC G01N 33/57438; C07K 16/2851; C07K 16/2818; G01N 33/57419; C07K 2317/24; G01N 33/57423; G01N 2333/4724; C07K 2317/565; G01N 33/57415

(57) ABSTRACT

Disclosed herein are methods for identifying a subject as having certain solid cancers such as breast cancer, colorectal colon cancer, or non-small cell lung cancer, or being at risk for the cancer based on the level of Galectin-9 in a biological sample (e.g., a blood sample) from a subject suspected of having the solid cancer.

### RELATED APPLICATION

This application claims the benefit of U.S. Provisional Application No. 62/841,732, filed on May 1, 2019, which is incorporated by reference herein in its entirety.

### BACKGROUND OF INVENTION

Galectin-9 is a tandem-repeat lectin consisting of two carbohydrate recognition domains (CRDs) and was discovered and described for the first time in 1997 in patients suffering from Hodgkin's lymphoma (HL) (Tureci et al., J. Biol. Chem. 1997, 272, 6416-6422). Three isoforms exist, and can be located within the cell or extracellularly. Elevated Galectin-9 levels have been in observed a wide range of cancers, including melanoma, Hodgkin's lymphoma, hepatocellular, pancreatic, gastric, colon and clear cell renal cell cancers (Wdowiak et al. Int. J. Mol. Sci. 2018, 19, 210). In renal cancer, patients with high Galectin-9 expression showed more advanced progression of the disease with larger tumor size (Kawashima et al.; BJU Int. 2014; 113:320-332). In melanoma, galectin-9 was expressed in 57% of tumors and was significantly increased in the plasma of patients with advanced melanoma compared to healthy controls (Enninga et al., Melanoma Res. 2016 October; 26(5): 429-441).

Galectin-9 has been described to play an important role in in a number of cellular processes such as adhesion, cancer cell aggregation, apoptosis, and chemotaxis. Recent studies have shown a role for Galectin-9 in immune modulation in support of the tumor, e.g., through negative regulation of Th1 type responses, Th2 polarization and polarization of macrophages to the M2 phenotype. This work also includes studies that have shown that Galectin-9 participates in direct inactivation of T cells through interactions with the T-cell immunoglobulin and mucin protein 3 (TIM-3) receptor (Dardalhon et al., J Immunol., 2010, 185, 1383-1392; Sanchez-Fueyo et al., Nat Immunol., 2003, 4, 1093-1101).

Galectin-9 has also been found to play a role in polarizing T cell differentiation into tumor suppressive phenotypes), as well as promoting tolerogenic macrophage programming and adaptive immune suppression (Daley et al., Nat Med., 2017, 23, 556-567). In mouse models of pancreatic ductal adenocarcinoma (PDA), blockade of the checkpoint interaction between Galectin-9 and the receptor Dectin-1 found on innate immune cells in the tumor microenvironment (TME) has been shown to increase anti-tumor immune responses in the TME and to slow tumor progression (Daley et al., Nat Med., 2017, 23, 556-567). Galectin-9 also has been found to bind to CD206, a surface marker of M2 type macrophages, resulting in a reduced secretion of CVL22 (MDC), a macrophage derived chemokine which has been associated with longer survival and lower recurrence risk in lung cancer (Enninga et al, J Pathol. 2018 August; 245(4):468-477).

### SUMMARY OF INVENTION

The present disclosure is based, at least in part, on the unexpected discovery that an elevated level of blood galectin-9 was observed in patients having certain solid cancers (e.g., breast cancer, colorectal cancer, and non-small cell lung cancer) as compared with the blood level of galectin-9 in healthy controls. As such, the level of galectin-9 can serve as a reliable biomarker for diagnosing such solid cancers.

Accordingly, one aspect of the present disclosure provides a method for identifying a cancer patient and optionally treating the cancer patient. In some examples, the cancer patient is a human cancer patient. In some embodiments, the method comprises: (i) providing a biological sample of a subject suspected of having a cancer, (ii) measuring the level of galaectin-9 in the biological sample (e.g., by an immunoassay), and (iii) identifying the subject as having the cancer or at risk for the cancer based on the level of galectin-9 in the biological sample. An elevated level of galectin-9 in the biological sample of the subject relative to a predetermined reference level indicates that the subject has the cancer or is at risk for the cancer. In some embodiments, the cancer is a solid cancer. In some examples, the solid cancer is breast cancer. In some examples, the solid cancer is colorectal colon cancer (CRC). In some examples, the solid cancer is non-small cell lung cancer (NSCLC). In some embodiments, the solid cancer is pancreatic cancer. In some embodiments, the cancer is metastatic cancer.

In some embodiments, the biological sample is a blood sample. For example, the blood sample can be a serum sample. In other examples, the blood sample can be a plasma sample. In some embodiments, the biological sample is a tissue sample, e.g., from a tumor biopsy. In some embodiments, the biological sample is derived from patient-derived organotypic tumor spheroids (PDOTs).

In some embodiments, any of the methods disclosed herein may further comprise determining tumor burden of the subject based on the level of galectin-9 in the biological sample. In some specific embodiments, any of the methods disclosed herein may further comprise determining tumor burden of a subject having pancreatic cancer based on the level of galectin-9 in the biological sample. Alternatively or in addition, the method may further comprise determining metastasis status of the cancer in the subject based on the level of galectin-9 in the biological sample. In some embodiments, the method further comprises determining metastasis status of the cancer in the subject based on the level of galectin-9 in the biological sample. In some embodiments, the method may further comprise performing one or more additional diagnostic assays to confirm occurrence of the cancer, tumor burden of the cancer, and/or metastatic status of the cancer. In some embodiments, any of the methods disclosed herein may further comprise performing an anti-cancer therapy to the subject for treating the cancer. In some instances, the anti-cancer therapy comprises administering to the subject an effective amount of an anti-Galectin-9 antibody. In some examples, the anti-Galectin-9 antibody comprises the same heavy chain complementary determining regions (CDRs) and the same light chain CDRs as reference antibody G9.2-17. In some embodiments, the anti-Galectin-9 antibody comprises a light chain complementarity determining region 1 (CDR1) set forth as SEQ ID NO: 1, a light chain complementary determining region 2 (CDR2) set forth as SEQ ID NO: 2, and a light chain complementary determining region 3 (CDR3) set forth as SEQ ID NO: 3 and/or comprises a heavy chain complementarity determining region 1 (CDR1) set forth as SEQ ID NO: 4, a heavy chain complementary determining region 2 (CDR2) set forth as SEQ ID NO: 5, and a heavy chain complementary determining region 3 (CDR3) set forth as SEQ ID NO: 6. In some embodiments, the anti-Galectin-9 antibody comprises a light chain complementarity determining region 1 (CDR1) set forth as SEQ ID NO: 1, a light chain complementary determining region 2 (CDR2) set forth as SEQ ID NO: 2, and a light chain complementary determining region 3 (CDR3) set forth as SEQ ID NO: 3 and comprises a heavy chain complementarity determining region 1 (CDR1) set forth as SEQ ID NO: 4, a heavy chain complementary determining region 2 (CDR2) set forth as SEQ ID NO: 5, and a heavy chain complementary determining region 3 (CDR3) set forth as SEQ ID NO: 6. In some examples, the anti-Galectin-9 antibody comprises a light chain variable domain of SEQ ID NO: 8, and/or a heavy chain variable domain of SEQ ID NO: 7. In some examples, the anti-Galectin-9 antibody comprises a light chain variable domain of SEQ ID NO: 8, and a heavy chain variable domain of SEQ ID NO: 7. Any of the anti-Galectin-9 antibodies disclosed herein may be a full-length antibody, for example, an IgG1 or IgG4 molecule. In specific examples, the anti-Galectin-9 antibody comprises a heavy chain comprising the amino acid sequence of SEQ ID NO:19 and a light chain comprising the amino acid sequence of SEQ ID NO:15. In some embodiments, the anti-cancer therapy comprises a checkpoint inhibitor, for example, an anti-PD-1 antibody or an anti-PD-L1 antibody.

One aspect of the present disclosure provides a method of diagnosing and optionally treating a cancer in a subject, e.g., a human subject. In some embodiments, the method comprises (i) providing a biological sample of a subject suspected of having a cancer, (ii) measuring the level of galectin-9 in the biological sample, and (iii) identifying the subject as having the cancer or being at risk for the cancer based on the level of galectin-9 in the biological sample. In some embodiments an elevated level of galectin-9 in the biological sample of the subject relative to a predetermined reference level indicates that the subject has the cancer or is at risk for the cancer. In some embodiments, the cancer is breast cancer. In some embodiments, the cancer is colorectal colon cancer (CRC). In some embodiments, the cancer is pancreatic cancer. In some embodiments, the cancer is non-small cell lung cancer (NSCLC). In some embodiments, the biological sample is a blood sample, e.g., a serum sample or a plasma sample. In some embodiments, the biological sample is a tissue sample, e.g., from a tumor biopsy. In some embodiments, the biological sample is derived from patient-derived organotypic tumor spheroids (PDOTs). In some embodiments, the level of galectin-9 is measured by an immunoassay. In some embodiments, the method further comprises determining tumor burden of the subject based on the level of galectin-9 in the biological sample. In some embodiments, the method further comprises determining metastatic status of the cancer in the subject based on the level of galectin-9 in the biological sample. In one specific embodiment, the cancer is pancreatic cancer. In some embodiments, the method further comprises: performing one or more additional diagnostic assays to confirm occurrence of the cancer or tumor burden of the cancer. In some embodiments, the method further comprises: performing an anti-cancer therapy to the subject for treating the cancer. In some embodiments, the anti-cancer therapy comprises administering to the subject an effective amount of an anti-Galectin-9 antibody. In some embodiments, the anti-Galectin-9 antibody comprises a light chain complementarity determining region 1 (CDR1) set forth as SEQ ID NO: 1, a light chain complementary determining region 2 (CDR2) set forth as SEQ ID NO: 2, and a light chain complementary determining region 3 (CDR3) set forth as SEQ ID NO: 3 and/or comprises a heavy chain complementarity determining region 1 (CDR1) set forth as SEQ ID NO: 4, a heavy chain complementary determining region 2 (CDR2) set forth as SEQ ID NO: 5, and a heavy chain complementary determining region 3 (CDR3) set forth as SEQ ID NO: 6. In some embodiments, the anti-Galectin-9 antibody comprises a light chain complementarity determining region 1 (CDR1) set forth as SEQ ID NO: 1, a light chain complementary determining region 2 (CDR2) set forth as SEQ ID NO: 2, and a light chain complementary determining region 3 (CDR3) set forth as SEQ ID NO: 3 and comprises a heavy chain complementarity determining region 1 (CDR1) set forth as SEQ ID NO: 4, a heavy chain complementary determining region 2 (CDR2) set forth as SEQ ID NO: 5, and a heavy chain complementary determining region 3 (CDR3) set forth as SEQ ID NO: 6. In some embodiments, the anti-Galectin-9 antibody comprises a light chain variable domain of SEQ ID NO: 8, and/or a heavy chain variable domain of SEQ ID NO: 7. In some embodiments, the anti-Galectin-9 antibody comprises a light chain variable domain of SEQ ID NO: 8, and a heavy chain variable domain of SEQ ID NO: 7. In some embodiments, the anti-Galectin-9 antibody is a full-length antibody. In some embodiments, the anti-Galectin-9 antibody is an IgG1 or IgG4 molecule. In some embodiments, the anti-Galectin-9 antibody comprises a heavy chain comprising the amino acid sequence of SEQ ID NO:19 and a light chain comprising the amino acid sequence of SEQ ID NO:15. In some embodiments, the anti-Galectin-9 antibody is G9.2-17 IgG4. In some embodiments, the anti-cancer therapy comprises a checkpoint inhibitor. In some embodiments, the checkpoint inhibitor is an anti-PD-1 antibody or an anti-PD-L1 antibody.

One aspect of the present disclosure provides a method for determining tumor burden or metastatic status in a cancer patient, e.g., a human cancer patient and optionally treating the cancer patient. In some embodiments, the method comprises: (i) providing a biological sample of a subject having a cancer, (ii) measuring the level of galectin-9 in the biological sample, and (iii) determining tumor burden of the subject based on the level of galectin-9 in the blood sample, wherein an elevated level of galectin-9 in the biological sample of the subject relative to a predetermined reference level indicates that the subject has a high tumor burden. In some embodiments, the cancer patient has breast cancer. In some embodiments, the patient has colorectal colon cancer (CRC). In some embodiments, the patient has pancreatic cancer. In some embodiments, the patient has non-small cell lung cancer (NSCLC). In some embodiments, the biological sample is a blood sample, e.g., a serum sample or a plasma sample. In some embodiments, the patient is a human subject. In some embodiments, the level of galectin-9 is measured by an immunoassay. In some embodiments, the method further comprises: performing one or more additional diagnostic assays to confirm occurrence of the cancer or tumor burden of the cancer. In some embodiments, method further comprises: performing an anti-cancer therapy to the subject for treating the cancer. In some embodiments, the anti-cancer therapy comprises administering to the subject an effective amount of an anti-Galectin-9 antibody. In some embodiments, the anti-Galectin-9 antibody comprises a light chain complementarity determining region 1 (CDR1) set forth as SEQ ID NO: 1, a light chain complementary determining region 2 (CDR2) set forth as SEQ ID NO: 2, and a light chain complementary determining region 3 (CDR3) set forth as SEQ ID NO: 3 and/or comprises a heavy chain complementarity determining region 1 (CDR1) set forth as SEQ ID NO: 4, a heavy chain complementary determining region 2 (CDR2) set forth as SEQ ID NO: 5, and a heavy chain complementary determining region 3 (CDR3) set forth as SEQ ID NO: 6. In some embodiments, the anti-Galectin-9 antibody comprises a light chain complementarity determining region 1 (CDR1) set forth as SEQ ID NO: 1, a light chain complementary determining region 2 (CDR2) set forth as SEQ ID NO: 2, and a light chain complementary determining region 3 (CDR3) set forth as SEQ ID NO: 3 and comprises a heavy chain complementarity determining region 1 (CDR1) set forth as SEQ ID NO: 4, a heavy chain complementary determining region 2 (CDR2) set forth as SEQ ID NO: 5, and a heavy chain complementary determining region 3 (CDR3) set forth as SEQ ID NO: 6.

In some embodiments, the anti-Galectin-9 antibody comprises a light chain variable domain of SEQ ID NO: 8, and/or a heavy chain variable domain of SEQ ID NO: 7. In some embodiments, the anti-Galectin-9 antibody comprises a light chain variable domain of SEQ ID NO: 8, and a heavy chain variable domain of SEQ ID NO: 7. In some embodiments, the anti-Galectin-9 antibody is a full-length antibody. In some embodiments, the anti-Galectin-9 antibody is an IgG1 or IgG4 molecule. In some embodiments, the anti-Galectin-9 antibody comprises a heavy chain comprising the amino acid sequence of SEQ ID NO:19 and a light chain comprising the amino acid sequence of SEQ ID NO:15. In some embodiments, the anti-Galectin-9 antibody is G9.2-17 IgG4. In some embodiments, the anti-cancer therapy comprises a checkpoint inhibitor. In some embodiments, the checkpoint inhibitor is an anti-PD-1 antibody or an anti-PD-L1 antibody.

One aspect of the present disclosure provides a method of treating a subject having cancer comprising (i) providing a biological sample of a subject suspected of having a cancer, (ii) measuring the level of galaectin-9 in the biological sample, and (iii) identifying the subject as having the cancer or being at risk for the cancer based on the level of galectin-9 in the biological sample, wherein an elevated level of galectin-9 in the biological sample of the subject relative to a predetermined reference level indicates that the subject has the cancer or is at risk for the cancer, and wherein the cancer is breast cancer, colorectal colon cancer (CRC), pancreatic cancer, or non-small cell lung cancer (NSCLC) and (iv) administering to the subject a therapeutically effective amount of a cancer therapy. In some embodiments, the biological sample is a blood sample, e.g., a serum sample or a plasma sample. In some embodiments, the biological sample is a tissue sample, e.g., from a tumor biopsy. In some embodiments, the biological sample is derived from patient-derived organotypic tumor spheroids (PDOTs). In some embodiments, the level of galectin-9 is measured by an immunoassay. In some embodiments, the method further comprises determining tumor burden of the subject based on the level of galectin-9 in the biological sample. In some embodiments, the method further comprises determining metastatic status of the cancer in the subject based on the level of galectin-9 in the biological sample. In one specific embodiment, the cancer is pancreatic cancer. In some embodiments, the method further comprises: performing one or more additional diagnostic assays to confirm occurrence of the cancer or tumor burden of the cancer. In some embodiments, the method further comprises: performing an anti-cancer therapy to the subject for treating the cancer. In some embodiments, the anti-cancer therapy comprises administering to the subject an effective amount of an anti-Galectin-9 antibody. In some embodiments, the anti-Galectin-9 antibody comprises a light chain complementarity determining region 1 (CDR1) set forth as SEQ ID NO: 1, a light chain complementary determining region 2 (CDR2) set forth as SEQ ID NO: 2, and a light chain complementary determining region 3 (CDR3) set forth as SEQ ID NO: 3 and/or comprises a heavy chain complementarity determining region 1 (CDR1) set forth as SEQ ID NO: 4, a heavy chain complementary determining region 2 (CDR2) set forth as SEQ ID NO: 5, and a heavy chain complementary determining region 3 (CDR3) set forth as SEQ ID NO: 6. In some embodiments, the anti-Galectin-9 antibody comprises a light chain complementarity determining region 1 (CDR1) set forth as SEQ ID NO: 1, a light chain complementary determining region 2 (CDR2) set forth as SEQ ID NO: 2, and a light chain complementary determining region 3 (CDR3) set forth as SEQ ID NO: 3 and comprises a heavy chain complementarity determining region 1 (CDR1) set forth as SEQ ID NO: 4, a heavy chain complementary determining region 2 (CDR2) set forth as SEQ ID NO: 5, and a heavy chain complementary determining region 3 (CDR3) set forth as SEQ ID NO: 6. In some embodiments, the anti-Galectin-9 antibody comprises a light chain variable domain of SEQ ID NO: 8, and/or a heavy chain variable domain of SEQ ID NO: 7. In some embodiments, the anti-Galectin-9 antibody comprises a light chain variable domain of SEQ ID NO: 8, and a heavy chain variable domain of SEQ ID NO: 7. In some embodiments, the anti-Galectin-9 antibody is a full-length antibody. In some embodiments, the anti-Galectin-9 antibody is an IgG1 or IgG4 molecule. In some embodiments, the anti-Galectin-9 antibody comprises a heavy chain comprising the amino acid sequence of SEQ ID NO:19 and a light chain comprising the amino acid sequence of SEQ ID NO:15. In some embodiments, the anti-Galectin-9 antibody is G9.2-17 IgG4. In some embodiments, the anti-cancer therapy comprises a checkpoint inhibitor. In some embodiments, the checkpoint inhibitor is an anti-PD-1 antibody or an anti-PD-L1 antibody.

In some specific embodiments, the disclosure provides methods for identifying a cancer patient and treating the cancer patient, the method comprising: (i) providing a biological sample of a subject suspected of having a cancer, (ii) measuring the level of galectin-9 in the biological sample, (iii) identifying the subject as having the cancer or being at risk for the cancer based on the level of galectin-9 in the biological sample, wherein an elevated level of galectin-9 in the biological sample of the subject relative to a predetermined reference level indicates that the subject has the cancer or is at risk for the cancer and wherein the cancer is breast cancer, colorectal colon cancer (CRC), pancreatic cancer, or non-small cell lung cancer (NSCLC), and (iv) administering to the subject a therapeutically effective amount of an anti-Galectin-9 antibody, wherein the anti-Galectin-9 antibody comprises a light chain complementarity determining region 1 (CDR1) set forth as SEQ ID NO: 1, a light chain complementary determining region 2 (CDR2) set forth as SEQ ID NO: 2, and a light chain complementary determining region 3 (CDR3) set forth as SEQ ID NO: 3 and/or comprises a heavy chain complementarity determining region 1 (CDR1) set forth as SEQ ID NO: 4, a heavy chain complementary determining region 2 (CDR2) set forth as SEQ ID NO: 5, and a heavy chain complementary determining region 3 (CDR3) set forth as SEQ ID NO: 6. In some of these specific embodiments, the anti-Galectin-9 antibody comprises a light chain complementarity determining region 1 (CDR1) set forth as SEQ ID NO: 1, a light chain complementary determining region 2 (CDR2) set forth as SEQ ID NO: 2, and a light chain complementary determining region 3 (CDR3) set forth as SEQ ID NO: 3 and comprises a heavy chain complementarity determining region 1 (CDR1) set forth as SEQ ID NO: 4, a heavy chain complementary determining region 2 (CDR2) set forth as SEQ ID NO: 5, and a heavy chain complementary determining region 3 (CDR3) set forth as SEQ ID NO: 6.

In some specific embodiments, the disclosure provides methods for diagnosing and treating a cancer in a subject, said method comprising (i) providing a biological sample of a subject suspected of having a cancer, (ii) measuring the level of galectin-9 in the biological sample, (iii) identifying the subject as having the cancer or being at risk for the cancer based on the level of galectin-9 in the biological sample, wherein an elevated level of galectin-9 in the biological sample of the subject relative to a predetermined reference level indicates that the subject has the cancer or is at risk for the cancer and wherein the cancer is breast cancer, colorectal colon cancer (CRC), pancreatic cancer, or non-small cell lung cancer (NSCLC), and (iv) administering to the subject a therapeutically effective amount of an anti-Galectin-9 antibody, wherein the anti-Galectin-9 antibody comprises a light chain complementarity determining region 1 (CDR1) set forth as SEQ ID NO: 1, a light chain complementary determining region 2 (CDR2) set forth as SEQ ID NO: 2, and/or a light chain complementary determining region 3 (CDR3) set forth as SEQ ID NO: 3 and comprises a heavy chain complementarity determining region 1 (CDR1) set forth as SEQ ID NO: 4, a heavy chain complementary determining region 2 (CDR2) set forth as SEQ ID NO: 5, and a heavy chain complementary determining region 3 (CDR3) set forth as SEQ ID NO: 6. In some of these specific embodiments, the anti-Galectin-9 antibody comprises a light chain complementarity determining region 1 (CDR1) set forth as SEQ ID NO: 1, a light chain complementary determining region 2 (CDR2) set forth as SEQ ID NO: 2, and a light chain complementary determining region 3 (CDR3) set forth as SEQ ID NO: 3 and comprises a heavy chain complementarity determining region 1 (CDR1) set forth as SEQ ID NO: 4, a heavy chain complementary determining region 2 (CDR2) set forth as SEQ ID NO: 5, and a heavy chain complementary determining region 3 (CDR3) set forth as SEQ ID NO: 6.

In some specific embodiments, the disclosure provides methods for treating a solid tumor in a subject comprising:

(a) providing a biological sample of a subject suspected of having a cancer,

(b) determining from the sample whether the subject has an increased level of galectin-9 relative to a reference or a healthy subject, and

(c) if the subject has a increased level of galectin-9 relative to a reference or a healthy subject, then administering to the subject an effective amount of an anti-Galectin-9 antibody,

In some embodiments, the cancer is breast cancer. In some embodiments, the cancer is colorectal colon cancer (CRC). In some embodiments, the cancer is pancreatic cancer. In some embodiments, the cancer is non-small cell lung cancer (NSCLC). In some embodiments, the biological sample is a blood sample, e.g., a serum sample or a plasma sample. In some embodiments, the biological sample is a tissue sample, e.g., from a tumor biopsy. In some embodiments, the biological sample is derived from patient-derived organotypic tumor spheroids (PDOTs). In some embodiments, the anti-Galectin-9 antibody comprises a light chain complementarity determining region 1 (CDR1) set forth as SEQ ID NO: 1, a light chain complementary determining region 2 (CDR2) set forth as SEQ ID NO: 2, and a light chain complementary determining region 3 (CDR3) set forth as SEQ ID NO: 3 and/or comprises a heavy chain complementarity determining region 1 (CDR1) set forth as SEQ ID NO: 4, a heavy chain complementary determining region 2 (CDR2) set forth as SEQ ID NO: 5, and a heavy chain complementary determining region 3 (CDR3) set forth as SEQ ID NO: 6. In some embodiments, the anti-Galectin-9 antibody comprises a light chain complementarity determining region 1 (CDR1) set forth as SEQ ID NO: 1, a light chain complementary determining region 2 (CDR2) set forth as SEQ ID NO: 2, and a light chain complementary determining region 3 (CDR3) set forth as SEQ ID NO: 3 and comprises a heavy chain complementarity determining region 1 (CDR1) set forth as SEQ ID NO: 4, a heavy chain complementary determining region 2 (CDR2) set forth as SEQ ID NO: 5, and a heavy chain complementary determining region 3 (CDR3) set forth as SEQ ID NO: 6. In some embodiments, the anti-Galectin-9 antibody comprises a light chain variable domain of SEQ ID NO: 8, and/or a heavy chain variable domain of SEQ ID NO: 7. In some embodiments, the anti-Galectin-9 antibody comprises a light chain variable domain of SEQ ID NO: 8, and a heavy chain variable domain of SEQ ID NO: 7. In some embodiments, the anti-Galectin-9 antibody is a full-length antibody. In some embodiments, the anti-Galectin-9 antibody is an IgG1 or IgG4 molecule. In some embodiments, the anti-Galectin-9 antibody comprises a heavy chain comprising the amino acid sequence of SEQ ID NO:19 and a light chain comprising the amino acid sequence of SEQ ID NO:15. In some embodiments, the anti-Galectin-9 antibody is G9.2-17 IgG4. In some embodiments, the anti-cancer therapy comprises a checkpoint inhibitor. In some embodiments, the checkpoint inhibitor is an anti-PD-1 antibody or an anti-PD-L1 antibody.

In some specific embodiments, the disclosure provides methods for treating a subject having a solid tumor comprising: (i) providing a biological sample of a subject suspected of having a cancer, (ii) measuring the level of galectin-9 in the biological sample, (iii) identifying the subject as having the cancer or being at risk for the cancer based on the level of galectin-9 in the biological sample, wherein an elevated level of galectin-9 in the biological sample of the subject relative to a predetermined reference level indicates that the subject has the cancer or is at risk for the cancer and wherein the cancer is breast cancer, colorectal colon cancer (CRC), pancreatic cancer, or non-small cell lung cancer (NSCLC), and (iv) administering to the subject a therapeutically effective amount of an anti-Galectin-9 antibody, wherein the anti-Galectin-9 antibody comprises a light chain complementarity determining region 1 (CDR1) set forth as SEQ ID NO: 1, a light chain complementary determining region 2 (CDR2) set forth as SEQ ID NO: 2, and/or a light chain complementary determining region 3 (CDR3) set forth as SEQ ID NO: 3 and comprises a heavy chain complementarity determining region 1 (CDR1) set forth as SEQ ID NO: 4, a heavy chain complementary determining region 2 (CDR2) set forth as SEQ ID NO: 5, and a heavy chain complementary determining region 3 (CDR3) set forth as SEQ ID NO: 6. In some of these specific embodiments, the anti-Galectin-9 antibody comprises a light chain complementarity determining region 1 (CDR1) set forth as SEQ ID NO: 1, a light chain complementary determining region 2 (CDR2) set forth as SEQ ID NO: 2, and a light chain complementary determining region 3 (CDR3) set forth as SEQ ID NO: 3 and comprises a heavy chain complementarity determining region 1 (CDR1) set forth as SEQ ID NO: 4, a heavy chain complementary determining region 2 (CDR2) set forth as SEQ ID NO: 5, and a heavy chain complementary determining region 3 (CDR3) set forth as SEQ ID NO: 6.

In any of the methods disclosed above, the predetermined reference level may represent the level of Galectin-9 in a biological sample (e.g., the same type of biological sample, such as a blood sample or tissue sample) from a control subject of the same species (e.g., a human subject) who is free of the target cancer. In some embodiments, the predetermined reference level refers to a pre-determined reference range of values representing the level of Galectin-9 in control subjects of the same species (e.g., human subjects) who are free of the target cancer, preferably free of any cancer. In some embodiments, the reference level is derived from a healthy tissue that is adjacent to a tumor. In any of the methods disclosed above the tumor may be metastatic.

In some embodiments, any of the methods described herein comprising the steps of (i) providing a biological sample of a subject suspected of having a cancer, and (ii) measuring the level of galectin-9 in the biological sample, are carried out separately from the body, e.g. are carried out on an in vitro tissue sample, e.g., blood or tumor tissue samples.

Thus, in some specific embodiments, the disclosure provides in vitro methods for identifying a cancer patient, the method comprising: (i) providing a biological sample of a subject suspected of having a cancer, (ii) measuring the level of galectin-9 in the biological sample in vitro, (iii) identifying the subject as having the cancer or being at risk for the cancer based on the level of galectin-9 in the invitro biological sample, wherein an elevated level of galectin-9 in the biological sample of the subject relative to a predetermined reference level indicates that the subject has the cancer or is at risk for the cancer and wherein the cancer is breast cancer, colorectal colon cancer (CRC), pancreatic cancer, or non-small cell lung cancer (NSCLC), and (iv) administering to the subject a therapeutically effective amount of an anti-Galectin-9 antibody, wherein the anti-Galectin-9 antibody comprises a light chain complementarity determining region 1 (CDR1) set forth as SEQ ID NO: 1, a light chain complementary determining region 2 (CDR2) set forth as SEQ ID NO: 2, and a light chain complementary determining region 3 (CDR3) set forth as SEQ ID NO: 3 and/or comprises a heavy chain complementarity determining region 1 (CDR1) set forth as SEQ ID NO: 4, a heavy chain complementary determining region 2 (CDR2) set forth as SEQ ID NO: 5, and a heavy chain complementary determining region 3 (CDR3) set forth as SEQ ID NO: 6. In some of these specific embodiments, the anti-Galectin-9 antibody comprises a light chain complementarity determining region 1 (CDR1) set forth as SEQ ID NO: 1, a light chain complementary determining region 2 (CDR2) set forth as SEQ ID NO: 2, and a light chain complementary determining region 3 (CDR3) set forth as SEQ ID NO: 3 and comprises a heavy chain complementarity determining region 1 (CDR1) set forth as SEQ ID NO: 4, a heavy chain complementary determining region 2 (CDR2) set forth as SEQ ID NO: 5, and a heavy chain complementary determining region 3 (CDR3) set forth as SEQ ID NO: 6.

Another aspect of the present disclosure provides a method for analyzing a biological sample from a subject suspected of having a cancer, the method comprising: (i) providing a biological sample of a subject suspected of having a cancer; and (ii) measuring a level of Galactin-9 in the biological sample with an antibody that specifically binds Galactin-9. In some embodiments, the subject is suspected of having a metastatic solid tumor. Examples include, but are not limited to, pancreatic adenocarcinoma (PDA), colorectal cancer (CRC), hepatocellular carcinoma (HCC), and breast cancer. In some embodiments, the subject is suspected of having a metastatic solid tumor. Another aspect of the present disclosure comprises a substance or composition, e.g., an anti-Galectin-9 antibody described herein or a composition thereof for use in the methods described herein.

The details of one or more embodiments of the invention are set forth in the description below. Other features or advantages of the present invention are be apparent from the following drawing and detailed description of several embodiments, and also from the appended claims.

### BRIEF DESCRIPTION OF DRAWINGS

The following drawings form part of the present specification and are included to further demonstrate certain aspects of the present disclosure, which can be better understood by reference to the drawing in combination with the detailed description of specific embodiments presented herein.

FIGS. 1A-1F include bar graphs showing levels of Galectin-9 expression as measured in T cells (CD3⁺), macrophages (CD11b+,) and tumor cells (Epcam+) in S2 and S3 organoid fractions derived from a pancreatic adenocarcinoma biopsy using anti-Galectin-9 G9.2-17 Fab fragment and a commercially available anti-Galectin-9 antibody (9M1-3). S2 fraction: organoids. S3 fraction: single cells. Corresponding isotype for G9.2-17 Fab (“Fab isotype”) and “fluorescence minus one” (FMO) 9M1-3 (“Gal9 FMO”) were used as controls for specificity, background staining and fluorescence bleed through from other channels. FIG. 1A shows levels of Galectin-9 in CD3⁺ cells as measured in the S3 fraction. FIG. 1B shows levels of Galectin-9 in CD11b⁺ cells as measured in the S3 fraction. FIG. 1C shows levels of Galectin-9 in Epcam⁺ cells as measured in the S3 fraction. FIG. 1D shows levels of Galectin-9 in CD3⁺ cells as measured in the S2 fraction. FIG. 1E shows levels of Galectin-9 in CD11b⁺ cells as measured in the S2 fraction. FIG. 1F shows levels of Galectin-9 in Epcam⁺ cells as measured in the S2 fraction.

FIGS. 2A-2F include bar graphs showing levels of Galectin-9 expression as measured in T cells (CD3⁺), macrophages (CD11b+,) and tumor cells (Epcam+) in S2 and S3 organoid fractions derived from a colorectal carcinoma biopsy using anti-Galectin-9 G9.2-17 Fab fragment and a commercially available anti-Galectin-9 antibody (9M1-3). S2 fraction: organoids. S3 fraction: single cells. Corresponding isotype for G9.2-17 Fab (“Fab isotype”) and FMO 9M1-3 (“Gal9 FMO”) were used controls for specificity, background staining and fluorescence bleed through from other channels. FIG. 2A shows levels of Galectin-9 in CD3⁺ cells as measured in the S3 fraction. FIG. 2B shows levels of Galectin-9 in CD11b⁺ cells as measured in the S3 fraction. FIG. 2C shows levels of Galectin-9 in Epcam⁺ cells as measured in the S3 fraction. FIG. 2D shows levels of Galectin-9 in CD3⁺ cells as measured in the S2 fraction. FIG. 2E shows levels of Galectin-9 in CD11b⁺ cells as measured in the S2 fraction. FIG. 2F shows levels of Galectin-9 in Epcam⁺ cells as measured in the S2 fraction.

FIGS. 3A-3F include bar graphs showing levels of Galectin-9 expression as measured in T cells (CD3⁺), macrophages (CD11b+,) and tumor cells (Epcam+) in S2 and S3 organoid fractions derived from a second pancreatic adenocarcinoma biopsy using anti-Galectin-9 G9.2-17 Fab fragment and a commercially available Galectin-9 antibody (9M1-3). S2 fraction: organoids. S3 fraction: single cells. Corresponding isotype for G9.2-17 Fab (“Fab isotype”) and FMO 9M1-3 (“Gal9 FMO”) were used as controls for specificity, background staining and fluorescence bleed through from other channels. FIG. 3A shows levels of Galectin-9 in CD3⁺ cells as measured in the S3 fraction. FIG. 3B shows levels of Galectin-9 in CD11b⁺ cells as measured in the S3 fraction. FIG. 3C shows levels of Galectin-9 in Epcam⁺ cells as measured in the S3 fraction. FIG. 3D shows levels of Galectin-9 in CD3⁺ cells as measured in the S2 fraction. FIG. 3E shows levels of Galectin-9 in CD11b⁺ cells as measured in the S2 fraction. FIG. 3F shows levels of Galectin-9 in Epcam⁺ cells as measured in the S2 fraction.

FIG. 4 is a graph showing measurements of galectin-9 levels in patient sera. Galectin-9 levels in patient sera (sample size in parentheses) was measured using enzyme-linked immunosorbant assay (ELISA). The ELISA was run in sandwich format using the Human Galectin-9 PicoKine ELISA kit (Bolster Biological Technology). Patient serum galectin-9 levels were compared to healthy human serum controls. Statistical analysis performed by unpaired Student's t-test. (*p<0.05; **p<0.01; ***p<0.001; ****p<0.0001). Results show Galectin-9 levels in serum is significantly increased in cancer patients.

FIGS. 5A-5C include photographs of immunohistochemical analysis of various tumors using anti-Galectin-9 antibody 1G3. All magnifications are 200× FIG. 5A shows Chemotherapy treated colorectal cancer with heterogeneous intensity score 2 and 3 (moderate and high) Galectin-9 expression. Galectin-9 staining was observed at the cell membrane in particular; additionally, intraglandular macrophages are moderately positive and stromal reaction in tumor shows multinucleated macrophage giant cells with moderately strong Galectin-9 expression. FIG. 5B shows liver metastasis of colorectal carcinoma with high (intensity score 3) Galectin-9 expression. Staining is located on the membrane and in the cytoplasm. FIG. 5C shows Galectin-9 positive (intensity score 2) entrapped bile ducts and Galectin-9 negative cancer.

### DETAILED DESCRIPTION OF INVENTION

Galectin-9, a tandem-repeat lectin, is a beta-galactoside-binding protein, which has been shown to have a role in modulating cell-cell and cell-matrix interactions. The present disclosure is based, at least in part, on the unexpected discovery of galectin-9 (e.g., blood level of galectin-9) as a biomarker that is correlated with occurrence, tumor burden, and/or metastatic status of certain solid tumors. Across multiple cohorts, galectin-9 was significantly increased in blood samples of individuals with primary and metastatic pancreatic cancer, lung tumors, and colorectal carcinoma, compared to healthy individuals. Accordingly, in some embodiments of the disclosure, measurement of galectin-9 levels can be used as a biomarker, for example, to select or identify patients, or to diagnose patients.

The present disclosure is based, at least in part, on the surprising observation that the serum level of galectin-9 correlates with occurrence, tumor burden, and/or metastatic status of solid cancers, such as those reported in Example 3 below. See, e.g., Table 2 as compared with Table 3, and Tables 4 and 5 below. Thus, galectin-9 can serve as a reliable biomarker for diagnosing such solid cancers and/or assessing tumor burden and/or metastatic status of the cancers.

Accordingly, provided herein are methods for diagnosing subjects (e.g., human patients) for having a target solid cancer using galectin-9 as a biomarker, for example, the blood level of galectin-9 as a biomarker. Also provided herein are methods for assessing tumor burden and/or metastatic status of a solid cancer in a subject using galectin-9 as a biomarker, for example, the blood level of galectin-9 as a biomarker.

### I. Methods for Cancer Diagnosis using Galectin-9 as a Biomarker

In some aspects, provided herein are diagnostic methods for cancer by measurement of Galectin-9 (Gal-9) levels in a biological sample obtained from a subject in need thereof. The Galectin-9 levels can be used as a biomarker, for example, to identify a patient as having the target cancer, to measure tumor burden in the patient, and/or to assess metastatic status of the cancer.

As used herein, “biomarker” refers to a distinctive biological or biologically derived indicator of a process, event or conditions. In certain embodiments, the biomarker is a gene or gene product (i.e., a polypeptide). Biological Samples for Measurement of Galectin-9

Any of the methods disclosed herein involve one or more biological samples collected from a subject such as a human subject at one or more suitable time points for measurement of Galectin-9 levels. In some examples, the subject may be a human subject suspected of having or at risk for a target cancer, for example, a breast cancer, a colorectal cancer, or a non-small cell lung cancer. Additional target cancers include, but are not limited to, melanoma, ovarian cancer, and pancreatic cancer. Such a patient may be free of any prior anti-cancer therapy. Alternatively, the patient may be free of any anti-cancer therapy. In some instances, the patient may have previously undergone an anti-cancer therapy, for example, immunotherapies, checkpoint inhibitor therapies, chemotherapies, radiotherapies, surgery, and combinations thereof.

In some examples, the biological sample can be a blood sample such as a serum sample or a plasma sample. The Galectin-9 levels may refer to the total amount of Galectin-9 in such a sample. Alternatively, the Galectin-9 levels may refer to the amount of circulating Galectin-9 in the sample. In other examples, the Galectin-9 levels may refer to the amount of cell surface Galectin-9 (e.g., Galectin-9 on tumor cells or Galectin-9 on immune cells) in the sample. In other examples, the biological sample can be a tissue sample, for example, a tumor tissue sample. In some specific examples, the tissue sample is a patient derived organoid (PDO) sample. The Galectin-9 level in such sample may refer to the total amount of Galectin-9 in the sample. In some examples, the Galectin-9 level may refer to the amount of Galectin-9 on a specific type of cells, e.g., tumor cells or tumor infiltrated lymphocytes (TILs).

In some embodiments, the level of Galectin-9 refers to the protein level of Galectin-9 in a biological sample. In other embodiments, the level of Galectin-9 refers to the messenger RNA level of Galectin-9 in a biological sample.

Levels of Galectin-9 in any of the biological samples disclosed herein may be measured by conventional methods. In some instances, levels of Galectin-9 may be measured by an immune assay, which refers to a biochemical assay for determining the presence or concentration of a target molecule through the use of an antibody or an antigen. Examples include, but are not limited to, enzyme-linked immunosorbent assays (ELISAs), Westernblot, radioimmunoassays (RIA), counting immunoassays (CIA), fluoroimmunoassays (FIA), and chemiluminescenceimmunoassays (CLIA). In some instances, flow cytometry may be used for measuring Galectin-9 positive cells in a sample. In some instances, immunohistochemistry may be used for measuring Galectin-9 positive cells in a sample. Other emerging protein analysis techniques which may be used are extensively known in the art (see e.g., Powers and Palecek, Protein analytical assays for diagnosing, monitoring, and choosing treatment for cancer patients J Healthc Eng. 2012 December; 3(4): 503-534) and include mass spectrometric techniques, such as matrix assisted laser desorption/ionization and surface enhanced laser desorption/ionization mass spectroscopy. Minaturization can be accomplished using techniques known in the art, such as using microfluidics. For example, the combination of microfluidics with traditional immunoassays, including IHC, flow cytometry, and ELISA, reduces antibody consumption by several orders of magnitude and offers the potential for assay automation. Additional assay tools include nanoparticles, e.g., polystyrene beads, quantum dots, gold particles or carbon nanotubes.

Levels of Galectin-9 nucleic acid levels, e.g., mRNA levels may be measured according to methods known in the art, e.g., using PCR-based techniques.

In some embodiments, levels of Galectin-9 expressed on the surface of cells derived from the human patient (e.g., from the blood or tumor of the human patient) are measured and compared to a reference level. In some embodiments, Galectin-9 expressed on the surface of cancer or or immune cells derived from corresponding a patient (e.g., macrophages, alpha/beta T cells or gamma/delta T cells), e.g., derived from a biopsy, PDOTs, or a blood sample, are measured and compared to a reference level.

B. Cancer Diagnosis

In some embodiments, provided here is a method for diagnosing a patient, for example, determining occurrence, tumor burden, and/or metastatic risk/status of a target cancer (e.g., those disclosed herein such as breast cancer, pancreatic cancer, CRC, and NSCLC) in a subject, such as a human patient.

Colorectal cancer (CRC), also known as bowel cancer, colon cancer, or rectal cancer, is any cancer affecting the colon and the rectum. Colorectal cancer presents one of the largest cancer burdens in the world, with approximately 700,000 people diagnosed globally each year. Despite significant advances in standard of care therapies, the five-year survival rate for metastatic colorectal cancer (CRC), remains around 12 percent. Death from CRC is expected to nearly double within the next 20 years. The current standard of care for CRC are chemotherapy regimens, combined and/or in sequence with anti-angiogenic therapy and anti-EGFR modalities. In addition, current immunotherapies are only efficacious (albeit producing profound and durable responses) in less than 20% of patients whose tumors demonstrate mismatch repair deficiency. CRC is known to be driven by genetic alterations of tumor cells and is also influenced by tumor-host interactions. Recent reports have demonstrated a direct correlation between the densities of certain T lymphocyte subpopulations and a favorable clinical outcome in CRC, supporting a major role of T-cell-mediated immunity in repressing tumor progression of CRC.

Breast cancer is one of the most common cancers that occur in women. There are many different types of breast cancer and common ones include ductal carcinoma in situ (DCIS) and invasive carcinoma. Others, like phyllodes tumors and angiosarcoma are less common. Breast cancer can spread when the cancer cells get into the blood or lymph system and are carried to other parts of the body. If cancer cells have spread to the lymph nodes, there is a higher chance that the cells could have traveled through the lymph system metastasized to other parts of the body.

Non-small cell lung cancer (NSCLC), which makes up the majority of lung cancers, is a disease in which malignant (cancer) cells form in the tissues of the lung. Non-small cell lung cancer is a group of lung cancer subtypes that behave similarly, such as adenocarcinoma, squamous cell carcinoma, adenosquamous carcinoma, sarcomatoid carcinoma, and large cell carcinoma. These subtypes, which start from different types of lung cells are grouped together as NSCLC because their treatment and prognoses (outlook) are often similar.

Pancreatic ductal adenocarcinoma (PDA) is a devastating disease with few long-term survivors (Yadav et al., Gastroenterology, 2013, 144, 1252-1261). Pancreatic cancer remains a disease that is difficult to treat due to a typically late presentation, relatively high resistance to chemotherapy, and lack of effective immune and targeted therapies. Globally, approximately 455,000 new cases of pancreatic cancer have been reported in 2018, and an estimated 355,000 new cases are estimated to occur until 2040 annually, and almost as many deaths are reported as new cases on a yearly basis. It is projected to be the second leading cause of cancer-related deaths in the United States by the year 2030. Despite intervention, the median life expectancy for patients with metastatic pancreatic cancer is less than 1 year with current treatment, while most patients (as many as 80%) present at an advanced/metastatic stage, when the disease is beyond curative resection. Despite advancements in the detection and management of pancreatic cancer, the five-year survival rate of metastatic disease remains at ten percent.

In some instances, the diagnosis is based on levels of Galectin-9 measured in a biological sample (e.g., those disclosed herein) collected from a subject, e.g., a human subject. In some embodiments, galectin-9 levels are measured in serum or plasma of a patient. In some embodiments, galectin-9 levels are measured in a tissue sample from the patient.

Accordingly, in some examples, the method comprises: (i) providing a biological sample such as a blood sample (e.g., a plasma sample or a serum sample) of a subject, e.g., a human subject, in need thereof, (ii) measuring the level of galectin-9 in the blood sample, and (iii) identifying the subject as having a cancer or being at risk for the cancer based on the level of galectin-9 in the blood sample. An elevated level of galectin-9 in the biological sample (e.g., a blood sample such as a serum sample or a plasma sample) of the subject relative to a predetermined reference level may indicate that the subject has the cancer or is at risk for the cancer.

In other examples, the method comprises: (i) providing a biological sample such as a blood sample (e.g., a plasma sample or a serum sample) of a subject, e.g., a human subject, in need thereof, (ii) measuring the level of galectin-9 in the blood sample, and (iii) assessing tumor burden of the subject based on the level of galectin-9 in the blood sample. An elevated level of galectin-9 in the biological sample (e.g., a blood sample such as a serum sample or a plasma sample) of the subject relative to a predetermined reference level may indicate that the subject has a high tumor burden.

In other examples, the method comprises: (i) providing a biological sample such as a blood sample (e.g., a plasma sample or a serum sample) of a subject, e.g., a human subject, in need thereof, (ii) measuring the level of galectin-9 in the blood sample, and (iii) assessing metastatic status of the subject based on the level of galectin-9 in the blood sample. An elevated level of galectin-9 in the biological sample (e.g., a blood sample such as a serum sample or a plasma sample) of the subject relative to a predetermined reference level may indicate that the subject has a metastatic cancer, e.g., a metastatic breast cancer, a metastatic CRC, or a metastatic NSCLC.

In some examples, the methods disclosed herein may comprise (i) providing a biopsy sample of a subject, e.g., a human subject, in need thereof, (ii) measuring the level of galectin-9 in the biopsy sample from a subject or in a sample derived from a biological sample of a subject, e.g., patient-derived organotypic tumor spheroids (PDOTs), e.g., prepared as described herein, and (iii) identifying the subject as having a cancer or being at risk for the cancer based on the level of galectin-9 in the sample. In some embodiments, the elevated level of galectin-9 in the sample of the subject is relative to a control and indicates that the subject has the cancer or is at risk for the cancer. In some embodiments, the control is cancer negative tissue from the same subject. In some embodiments, the control is a reference value or range of values. In some embodiments, the control is derived from a healthy subject. In some embodiments, the measuring involves determining levels of Galectin-9 protein or gene expression, e.g., mRNA levels.

In any of the methods disclosed above, the predetermined reference level may represent the level of Galectin-9 in a biological sample (e.g., the same type of biological sample, such as a blood sample) from a control subject of the same species (e.g., a human subject) who is free of the target cancer. Preferably, the control subject is free of any type of cancer. In some embodiments, the predetermined reference level refers to a pre-determined reference range of values representing the level of Galectin-9 in control subjects of the same species (e.g., human subjects) who are free of the target cancer, preferably free of any cancer. The control subjects may have matched physiological features as the subject, for example, age, gender, ethnic background, etc. Accordingly, levels higher than the reference values are indicative of an increased level. If the Galectin-9 level in the biological sample of a candidate subject (e.g., a human subject) is elevated relative to the predetermined reference level, this indicates that the candidate subject has or is at risk for the target cancer as those disclosed herein.

In some embodiments, the predetermined reference level may represent the level of Galectin-9 in a biological sample (e.g., the same type of biological sample, such as a blood sample) from a control patient having the target cancer at a low tumor burden. The control patient may have matched physiological features as the subject, for example, age, gender, ethnic background, etc. Accordingly, levels higher than the reference values are indicative of an increased level. If the Galectin-9 level in the biological sample of a candidate subject (e.g., a human subject) is elevated relative to the predetermined reference level, this indicates that the candidate subject has the target cancer as those disclosed herein at a high tumor burden. As used herein, tumor burden refers to amount of cancer, the size or the volume of the tumor in the body of a subject, accounting for all sites of disease. When a subject is identified as having a high tumor burden by a method disclosed herein, the tumor burden in the subject can be confirmed using methods known in the art, including but not limited to, FDG positron emission tomography (FDG-PET), magnetic resonance imaging (MM), and optical imaging, comprising bioluminescence imaging (BLI) and fluorescence imaging (FLI).

In some embodiments, the predetermined reference level may represent the level of Galectin-9 in a biological sample (e.g., the same type of biological sample, such as a blood sample) from a control patient having the target cancer without metastasis. The control patient may have matched physiological features as the subject, for example, age, gender, ethnic background, etc. Accordingly, levels higher than the reference values are indicative of an increased level. If the Galectin-9 level in the biological sample of a candidate subject (e.g., a human subject) is elevated relative to the predetermined reference level, this indicates that the candidate subject has the target cancer in metastatic status. As used herein, metastatic solid tumors/cancer refer to tumors/cancers having tumor/cancer cells from the place where they first form to another part of the body. In metastasis, cancer cells break away from the original tumor, travel through the blood or lymph system, and form a new tumor in other organs or tissues of the body.

In some embodiments, a Galectin-9 level may be deemed elevated as compared with a predetermined reference level when the Galectin-9 level is at least 20% higher, 30% higher, 40% higher, for example, at least 50% higher, at least 80% higher (including any numerical increment between the listed percentages), or at least 2-fold higher, than the predetermined reference level. In some embodiments, a Galectin-9 level may be deemed elevated as compared with a predetermined reference level when the Galectin-9 level is at least about 20% higher, 30% higher, 40% higher, for example, at least about 50% higher, at least about 80% higher (including any numerical increment between the listed percentages), or at least about 2-fold higher, than the predetermined reference level. In some embodiments, a Galectin-9 level may be deemed elevated as compared with a predetermined reference level when the Galectin-9 level is at least 2-fold higher, at least 3-fold higher, at least 4-fold higher, at least 5-fold higher, at least 6-fold higher, at least 7-fold higher, at least 8-fold higher, at least 9-fold higher, at least 10-fold higher, at least 10-fold to 15-fold higher, at least 15-fold to 20-fold higher, at least 20-fold to 25-fold higher, or at least 25-fold to 30-fold higher (including any numerical increment between the listed values). In some embodiments, a Galectin-9 level may be deemed elevated as compared with a predetermined reference level when the Galectin-9 level is at least about 2-fold higher, at least about 3-fold higher, at least about 4-fold higher, at least about 5-fold higher, at least about 6-fold higher, at least about 7-fold higher, at least about 8-fold higher, at least about 9-fold higher, at least about 10-fold higher, at least about 10-fold to 15-fold higher, at least about 15-fold to 20-fold higher, at least about 20-fold to 25-fold higher, or at least about 25-fold to 30-fold higher (including any numerical increment between the listed values).

In some embodiments, the methods of diagnosing a subject and optionally treating the subject described herein comprise a step (iii), wherein a Galectin-9 level is deemed elevated as compared with a predetermined reference level when the Galectin-9 level is at least 2-fold higher or at least about 2-fold higher. In some embodiments, the methods of diagnosing a subject and optionally treating the subject described herein comprise a step (iii), wherein a Galectin-9 level is deemed elevated as compared with a predetermined reference level when the Galectin-9 level is at least 2.5-fold higher or about 2.5-fold higher. In some embodiments, the methods of diagnosing a subject and optionally treating the subject described herein comprise a step (iii), wherein a Galectin-9 level is deemed elevated as compared with a predetermined reference level when the Galectin-9 level is at least 4-fold higher or at least about 4-fold higher. In some embodiments, the methods of diagnosing a subject and optionally treating the subject described herein comprise a step (iii), wherein a Galectin-9 level is deemed elevated as compared with a predetermined reference level when the Galectin-9 level is at least 20-fold higher or at least about 20-fold higher.

In some embodiments, the methods of identifying a cancer patient and optionally treating the cancer patient described herein comprise a step (iii), wherein a Galectin-9 level is deemed elevated as compared with a predetermined reference level when the Galectin-9 level is at least 2-fold higher or at least about 2-fold higher. In some embodiments, the methods of identifying a cancer patient and optionally treating the cancer patient described herein comprise a step (iii), wherein a Galectin-9 level is deemed elevated as compared with a predetermined reference level when the Galectin-9 level is at least 2.5-fold higher or about 2.5-fold higher. In some embodiments, the methods of identifying a cancer patient and optionally treating the cancer patient described herein comprise a step (iii), wherein a Galectin-9 level is deemed elevated as compared with a predetermined reference level when the Galectin-9 level is at least 4-fold higher or at least about 4-fold higher. In some embodiments, the methods of identifying a cancer patient and optionally treating the cancer patient described herein comprise a step (iii), wherein a Galectin-9 level is deemed elevated as compared with a predetermined reference level when the Galectin-9 level is at least 20-fold higher or at least about 20-fold higher.

In some embodiments, the methods of treating a subject having cancer described herein comprise a step (iii), wherein a Galectin-9 level is deemed elevated as compared with a predetermined reference level when the Galectin-9 level is at least 2-fold higher or at least about 2-fold higher. In some embodiments, the methods of treating a subject having cancer described herein comprise a step (iii), wherein a Galectin-9 level is deemed elevated as compared with a predetermined reference level when the Galectin-9 level is at least 2.5-fold higher or about 2.5-fold higher. In some embodiments, the methods of treating a subject having cancer described herein comprise a step (iii), wherein a Galectin-9 level is deemed elevated as compared with a predetermined reference level when the Galectin-9 level is at least 4-fold higher or at least about 4-fold higher. In some embodiments, the methods of treating a subject having cancer described herein comprise a step (iii), wherein a Galectin-9 level is deemed elevated as compared with a predetermined reference level when the Galectin-9 level is at least 20-fold higher or at least about 20-fold higher.

In some embodiments, the methods for determining tumor burden or metastatic status in a cancer patient and optionally treating the cancer patient described herein comprise a step (iii), wherein a Galectin-9 level is deemed elevated as compared with a predetermined reference level when the Galectin-9 level is at least 3-fold higher or at least about 3-fold higher. In some embodiments, the methods for determining tumor burden or metastatic status in a cancer patient and optionally treating the cancer patient described herein comprise a step (iii), wherein a Galectin-9 level is deemed elevated as compared with a predetermined reference level when the Galectin-9 level is at least 4-fold higher or about 4-fold higher. In some embodiments, the methods for determining tumor burden or metastatic status in a cancer patient and optionally treating the cancer patient described herein comprise a step (iii), wherein a Galectin-9 level is deemed elevated as compared with a predetermined reference level when the Galectin-9 level is at least 5-fold higher or at least about 5-fold higher. In some embodiments, the methods for determining tumor burden or metastatic status in a cancer patient and optionally treating the cancer patient described herein comprise a step (iii), wherein a Galectin-9 level is deemed elevated as compared with a predetermined reference level when the Galectin-9 level is at least 6-fold higher or at least about 6-fold higher.

In some examples, one or more other diagnostic tests known in the art can be used in conjunction with the method to confirm cancer occurrence, risk, tumor burden, and/or metastatic status. When a subject is identified as having the target cancer or at risk for the target cancer, a suitable anti-cancer therapy can be applied to such a patient. When a subject is identified as having the target cancer with a high tumor burden and/or with metastasis (or at risk for it), a suitable anti-cancer therapy can be selected based on the assessment. Details of anti-cancer therapies for treating any of the target cancers are provided below.

In some embodiments, the cancer to be diagnosed and/or treated is selected from adrenal cancer, adrenocortical carcinoma, anal cancer, appendix cancer, bile duct cancer, bladder cancer, bone cancer (e.g., Ewing sarcoma tumors, osteosarcoma, malignant fibrous histiocytoma), brain cancer (e.g., astrocytomas, brain stem glioma, craniopharyngioma, ependymoma), bronchial tumors, cholangiocarcinoma, cholangiosarcoma, central nervous system tumors, breast cancer, Castleman disease, cervical cancer, colon cancer, rectal cancer, colorectal cancer, endometrial cancer, esophageal cancer, eye cancer, gallbladder cancer, gastrointestinal cancer, gastrointestinal carcinoid tumors, gastrointestinal stromal tumors, genitourinary cancers, gestational trophoblastic disease, heart cancer, Kaposi sarcoma, kidney cancer, laryngeal cancer, hypopharyngeal cancer, leukemia (e.g., acute lymphoblastic leukemia, acute myeloid leukemia, chronic lymphocytic leukemia, chronic myelogenous leukemia), liver cancer, lung cancer (for example, non-small cell lung cancer, NSCLC, and small cell lung cancer, SCLC), lymphoma (e.g., AIDS-related lymphoma, Burkitt lymphoma, cutaneous T cell lymphoma, Hogkin lymphoma, Non-Hogkin lymphoma, primary central nervous system lymphoma), malignant mesothelioma, multiple myeloma, myelodysplastic syndrome, nasal cavity cancer, paranasal sinus cancer, pancreatic duct adenocarcinoma (PDA) nasopharyngeal cancer, neuroblastoma, oral cavity cancer, oropharyngeal cancer, osteosarcoma, ovarian cancer, pancreatic cancer, penile cancer, pituitary tumors, prostate cancer, retinoblastoma, rhabdomyosarcoma, rhabdoid tumor, salivary gland cancer, sarcoma, skin cancer (e.g., basal cell carcinoma, melanoma), squamous cell head and neck cancer, small intestine cancer, stomach cancer, teratoid tumor, testicular cancer, throat cancer, thymus cancer, thyroid cancer, unusual childhood cancers, upper and lower gastrointestinal malignancies (including, but not limited to, esophageal, gastric, and hepatobiliary cancer), urethral cancer, uterine cancer, uterine sarcoma, vaginal cancer, vulvar cancer, Waldenstrom macroglobulinemia, Wilms tumor and atypical theratoid rhabdoid tumor. In some embodiments, the cancer is selected from hematological malignancies including acute lymphoblastic leukemia, chronic lymphocytic leukemia, lymphomas, multiple myeloma, acute myelogenous leukemia, chronic myelogenous leukemia, myelodysplastic syndromes and the myeloproliferative neoplasms, such as essential thrombocythemia, polycythemia vera, myelofibrosis, and gallbladder cancer (adenocarcinomas or squamous cell carcinoma).

### II. Cancer Treatment

In other aspects, provided herein are methods for treating the cancer in a subject (e.g., a human patient) who is diagnosed as having the cancer (for example, having the cancer with a high tumor burden, or having the cancer with metastasis or at risk for such).

One or more suitable anti-cancer therapies may be selected based on the disease status of the patient to be treatment via routine medical practice. Examples include surgery, chemotherapy, radiation therapy, hormonal therapy (e.g., RDs, SERMs, and/or aromatase inhibitors), immunotherapy, complementary and holistic medicine, or a combination thereof. Examples include, but are not limited to, Abemaciclib, Abraxane®, Ado-Trastuzumab Emtansine Afinitor (Everolimus), Afinitor Disperz (Everolimus), Alpelisib, Anastrozole, Aredia (Pamidronate Disodium), Arimidex (Anastrozole), Aromasin (Exemestane), Atezolizumab, Capecitabine, Cyclophosphamide, Docetaxel, Doxorubicin Hydrochloride, Ellence (Epirubicin Hydrochloride), Enhertu (Fam-Trastuzumab Deruxtecan-nxki), Epirubicin Hydrochloride, Eribulin Mesylate, Everolimus, Exemestane, 5-FU (Fluorouracil Injection), Fam-Trastuzumab Deruxtecan-nxki, Fareston (Toremifene), Faslodex (Fulvestrant), Femara (Letrozole), Fluorouracil Injection, Fulvestrant, Gemcitabine Hydrochloride, Gemzar (Gemcitabine Hydrochloride), Goserelin Acetate, Halaven (Eribulin Mesylate), Herceptin Hylecta (Trastuzumab and Hyaluronidase-oysk), Herceptin (Trastuzumab), Ibrance (Palbociclib), Ixabepilone, Ixempra (Ixabepilone), Kadcyla (Ado-Trastuzumab Emtansine), Kisqali (Ribociclib), Lapatinib Ditosylate, Letrozole, Lynparza (Olaparib), Megestrol Acetate, Methotrexate, Neratinib Maleate, Nerlynx (Neratinib Maleate), Olaparib, Paclitaxel, Paclitaxel Albumin-stabilized Nanoparticle Formulation, Palbociclib, Pamidronate Disodium, Perjeta (Pertuzumab), Pertuzumab, Piqray (Alpelisib), Ribociclib, Talazoparib Tosylate, Talzenna (Talazoparib Tosylate), Tamoxifen Citrate, Taxol (Paclitaxel), Taxotere (Docetaxel), Tecentriq (Atezolizumab), Thiotepa, Toremifene, Trastuzumab, Trastuzumab and Hyaluronidase-oysk, Trexall (Methotrexate), Tykerb (Lapatinib Ditosylate), Verzenio (Abemaciclib), Vinblastine Sulfate, Xeloda (Capecitabine), and Zoladex (Goserelin Acetate). In some examples, the anti-cancer therapy involves an anti-Galectin-9 antibody. In some examples, the anti-cancer therapy involves an anti-PD1 antibody. In some examples, the anti-cancer therapy involves an anti-Galectin-9 antibody and an anti-PD1 antibody. Non-limiting examples of such PD-1 antibodies include pembrolizumab, nivolumab, tislelizumab and cemiplimab.

A. Anti-Galectin-9 Antibodies

Anti-Galectin-9 antibodies can serve as therapeutic agents for treating diseases associated with Galectin-9 (e.g., those in which a Galectin-9 signaling plays a role). Without being bound by theory, an anti-Galectin-9 antibody may block a signaling pathway mediated by Galectin-9. For example, the antibody may interfere with the interaction between Galectin-9 and its binding partner (e.g., Dectin-1, TIM-3 or CD206), thereby blocking the signaling triggered by the Galectin-9/Ligand interaction. Alternatively, or in addition, an anti-Galectin-9 antibody may also exert its therapeutic effect by inducing blockade and/or cytotoxicity, for example, ADCC, CDC, or ADCP against pathologic cells that express Galectin-9. A pathologic cell refers to a cell that contributes to the initiation and/or development of a disease, either directly or indirectly.

The anti-Galectin-9 antibodies disclosed herein are capable of suppressing the signaling mediated by Galectin-9 (e.g., the signaling pathway mediated by Galectin-9/Dectin-1 or Galectin-9/Tim-3) or eliminating pathologic cells expressing Galectin-9 via, e.g., ADCC. Accordingly, the anti-Galectin-9 antibodies described herein can be used for inhibiting any of the Galectin-9 signaling and/or eliminating Galectin-9 positive pathologic cells, thereby benefiting treatment of diseases associated with Galectin-9, for example, autoimmune diseases, infectious disorders, solid tumors and other cancers, allergic disorders, or hematological disorders such as hematological malignancies.

An antibody (interchangeably used in plural form) is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody”, e.g., anti-Galectin-9 antibody, encompasses not only intact (e.g., full-length) polyclonal or monoclonal antibodies, but also antigen-binding fragments thereof (such as Fab, Fab′, F(ab′)2, Fv), single chain (scFv), mutants thereof, fusion proteins comprising an antibody portion, humanized antibodies, chimeric antibodies, diabodies, nanobodies, linear antibodies, single chain antibodies, multispecific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies. An antibody, e.g., anti-Galectin-9 antibody, includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

A typical antibody molecule comprises a heavy chain variable region (V_{H}) and a light chain variable region (V_{L}), which are usually involved in antigen binding. The V_{H} and V_{L} regions can be further subdivided into regions of hypervariability, also known as “complementarity determining regions” (“CDR”), interspersed with regions that are more conserved, which are known as “framework regions” (“FR”). Each V_{H} and V_{L} is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the “Contact” numbering scheme, the IMGT” numbering scheme, the “AHo” numbering scheme, the Chothia definition, the AbM definition, the EU definition, and/or the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242, Chothia et al., (1989) Nature 342:877; Chothia, C. et al. (1987) J. Mol. Biol. 196:901-917; Al-lazikani et al (1997) J. Molec. Biol. 273:927-948; Edelman et al., Proc Natl Acad Sci US A. 1969 May; 63(1):78-85; Almagro, J. Mol. Recognit. 17:132-143 (2004); MacCallum et al., J. Mol. Biol. 262:732-745 (1996); Lefranc M P et al., Dev Comp Immunol, 2003 January; 27(1):55-77; and Honegger A and Pluckthun A, J Mol Biol, 2001 Jun. 8; 309(3):657-70). See also hgmp.mrc.ac.uk and bioinforg.uk/abs).

In some embodiments, the anti-Galectin-9 antibody described herein is a full-length antibody, which contains two heavy chains and two light chains, each including a variable domain and a constant domain. Alternatively, the anti-Galectin-9 antibody can be an antigen-binding fragment of a full-length antibody. Examples of binding fragments encompassed within the term “antigen-binding fragment” of a full length antibody include (i) a Fab fragment, a monovalent fragment consisting of the V_{L}, V_{H}, C_{L} and C_{H}1 domains; (ii) a F(ab′)₂ fragment, a bivalent fragment including two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR) that retains functionality. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules known as single chain Fv (scFv). See e.g., Bird et al. (1988) Science 242:423-426; and Huston et al. (1988) Proc. Natl. Acad. Sci. USA 85:5879-5883.

Any of the antibodies described herein, e.g., anti-Galectin-9 antibody, can be either monoclonal or polyclonal. A “monoclonal antibody” refers to a homogenous antibody population and a “polyclonal antibody” refers to a heterogeneous antibody population. These two terms do not limit the source of an antibody or the manner in which it is made.

In some examples, an anti-Galectin 9 antibody is G9.2-17 or a functional equivalent thereof. Reference antibody G9.2-17 refers to an antibody capable of binding to human Galectin-9 and comprises a heavy chain variable region of SEQ ID NO:7 and a light chain variable domain of SEQ ID NO:8, both of which are provided below. The CDRs provided in Table 1 and identified in the V_{H} and V_{L} sequences are based on the Kabat method.

TABLE 1
Heavy and Light Chain CDRs of G9.2-17

| | | | |
|---|---|---|---|
| G9.2-17 | V_{L} CDR1 | RASQSVSSAVA | SEQ ID NO: 1 |
| | V_{L} CDR2 | SASSLYS | SEQ ID NO: 2 |
| | V_{L} CDR3 | QQSSTDPIT | SEQ ID NO: 3 |
| | V_{H} CDR1 | FTVSSSSIH | SEQ ID NO: 4 |
| | V_{H} CDR2 | YISSSSGYTYYADSVKG | SEQ ID NO: 5 |
| | V_{H} CDR3 | YWSYPSWWPYRGMDY | SEQ ID NO: 6 |

V_{H}:
(SEQ ID NO: 7)
EVQLVESGGGLVQPGGSLRLSCAASGFTVSSSSIHWVRQAPGKGLEWVA
YISSSSGYTYYADSVKGRFTISADTSKNTAYLQMNSLRAEDTAVYYCAR
YWSYPSWWPYRGMDYWGQGTLVTVSS
V_{L}:
(SEQ ID NO: 8)
DIQMTQSPSSLSASVGDRVTITCRASQSVSSAVAWYQQKPGKAPKLLIY
SASSLYSGVPSRFSGSRSGTDFTLTISSLQPEDFATYYCQQSSTDPITF
GQGTKVEIKR

In some embodiments, the anti-Galectin-9 antibody for use in the methods disclosed herein is an antibody having the same heavy chain complementary determining regions (CDRs) as reference antibody G9.2-17 and/or the same light chain complementary determining regions as reference antibody G9.2-17. In some embodiments, the anti-Galectin-9 antibody for use in the method disclosed herein can be an antibody having the same heavy chain variable region (V_{H}) and/or the same light chain variable region (V_{L}) as reference antibody G9.2-17. Two antibodies having the same V_{H} and/or V_{L} CDRs means that their CDRs are identical when determined by the same approach (e.g., the Kabat approach, the Chothia approach, the AbM approach, the Contact approach, or the IMGT approach as known in the art. See, e.g., bioinf.org.uk/abs/). Exemplary numbering schemes for determining antibody CDRs include the “Kabat” numbering scheme (Kabat et al. (1991), 5th Ed. Public Health Service, National Institutes of Health, Bethesda, Md.), the “Chothia” numbering scheme (Al-Lazikani et al., (1997) JMB 273,927-948), the “Contact” numbering scheme (MacCallum et al., J. Mol. Biol. 262:732-745 (1996)), the “IMGT” numbering scheme (Lefranc M P et al., Dev Comp Immunol, 2003 January; 27(1):55-77), and the “AHo” numbering scheme (Honegger A and Pluckthun A, J Mol Biol, 2001 Jun. 8; 309(3):657-70).

In some instances, the anti-Galectin-9 antibody disclosed herein is a functional variant of reference antibody G9.2-17. A functional variant can be structurally similar as the reference antibody (e.g., comprising the limited number of amino acid residue variations in one or more of the heavy chain and/or light chain CDRs as G9.2-17 as disclosed herein, or the sequence identity relative to the heavy chain and/or light chain CDRs of G9.2-17, or the V_{H} and/or V_{L} of G9.2-17 as disclosed herein) with substantially similar binding affinity (e.g., having a KD value in the same order) to human Galectin-9.

In some embodiments, the anti-Galectin-9 antibody comprises heavy and light chain variable regions, wherein the light chain variable region CDR1, CDR2, and CDR3 amino acid sequences have at least 80% (e.g., 80%, 85%, 90%, 91%, 92%, 93%, 94%, 95%, 96%, 97%, 98%, or 99% and any increment therein) sequence identity to the light chain variable region CDR1, CDR2, and CDR3 amino acid sequences set forth in SEQ ID NOs: 1, 2, and 3, respectively. In some embodiments, the anti-Galectin-9 antibody comprises heavy and light chain variable regions, wherein the heavy chain variable region CDR1, CDR2, and CDR3 amino acid sequences have at least 80% (e.g., 80%, 85%, 90%, 91%, 92%, 93%, 94%, 95%, 96%, 97%, 98%, or 99% and any increment therein) sequence identity to the heavy chain variable region CDR1, CDR2, and CDR3 amino acid sequences set forth in SEQ ID NO: 4, 5, and 6, respectively.

The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and)(BLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of the invention. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

In other embodiments, the anti-Galectin-9 antibody described herein comprises a V_{H} that comprises the HC CDR1, HC CDR2, and HC CDR3, which collectively contain up to 8 amino acid residue variations (8, 7, 6, 5, 4, 3, 2, or 1 variations(s), including additions, deletions, and/or substitutions) relative to the HC CDR1, HC CDR2, and HC CDR3 of reference antibody G9.2-17. Alternatively or in addition, in some embodiments, the anti-Galectin-9 antibody described herein comprises a V_{L} that comprises the LC CDR1, LC CDR2, and LC CDR3, which collectively contain up to 8 amino acid residue variations (8, 7, 6, 5, 4, 3, 2, or 1 variations(s) including additions, deletions, and/or substitutions) relative to the LC CDR1, LC CDR2, and LC CDR3 of reference antibody G9.2-17.

In one example, the amino acid residue variations are conservative amino acid residue substitutions. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g., Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

Additional Galectin-9 antibodies, e.g., which bind to the CRD1 and/or CRD2 region of Galectin-9 are described U.S. Pat. No. 10,344,091 and WO 2019/084553, the relevant disclosures of each of which are incorporated by reference for the subject matter and purpose referenced herein.

The anti-Gal9 antibody, including the reference antibody G9.2-17, can be in any format as disclosed herein, for example, a full-length antibody or a Fab. In some embodiments, the heavy chain of any of any of the anti-Galectin-9 antibodies as described herein further comprise a heavy chain constant region (CH) or a portion thereof (e.g., CH1, CH2, CH3, or a combination thereof). The heavy chain constant region can be of any suitable origin, e.g., human, mouse, rat, or rabbit. In one specific example, the heavy chain constant region is from a human IgG (a gamma heavy chain) of any IgG subfamily as described herein.

In some embodiments, the heavy chain constant region of the antibodies described herein comprises a single domain (e.g., CH1, CH2, or CH3) or a combination of any of the single domains, of a constant region (e.g., SEQ ID NO: 10, 12, 13, 14, 20, and 21). In some embodiments, the light chain constant region of the antibodies described herein comprise a single domain (e.g., CL), of a constant region. Exemplary light and heavy chain sequences are listed below. Exemplary light and heavy chain sequences are listed below. The hIgG1 LALA sequence includes two mutations, L234A and L235A (EU numbering), which suppress FcgR binding as well as a P329G mutation (EU numbering) to abolish complement C1q binding, thus abolishing all immune effector functions. The hIgG4 Fab Arm Exchange Mutant sequence includes a mutation to suppress Fab Arm Exchange (S228P; EU numbering). An IL2 signal sequence (MYRMQLLSCIALSLALVTNS; SEQ ID NO: 9) can be located N-terminally of the variable region. It is used in expression vectors, which is cleaved during secretion and thus not in the mature antibody molecule. The mature protein (after secretion) starts with “EVQ” for the heavy chain and “DIM” for the light chain. Amino acid sequences of exemplary heavy chain constant regions are provided below:

hIgG1 Heavy Chain Constant Region
(SEQ ID NO: 10)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPELLGGPSVFLFPPKPKDTLMISRTPEVTCVVV
DVSHEDPEVKFNNYVDGVEVHNAKTKPREEQYNSTYRVVSVLTVLHQDW
LNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLT
VDKSRWQQGNVFSCSVMHEALHNHYTQKSLSLSPGK*
hIgG1 LALA Heavy Chain Constant Region
(SEQ ID NO: 12)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPEAAGGPSVFLFPPKPKDTLMISRTPEVTCVVV
DVSHEDPEVKFNNYVDGVEVHNAKTKPREEQYNSTYRVVSVLTVLHQDW
LNGKEYKCKVSNKALGAPIEKTISKAKGQPREPQVYTLPPSREEMTKNQ
VSLTCLVKGFYPSDIAVEWESNGQPENNYKTTPPVLDSDGSFFLYSKLT
VDKSRWQQGNVFSCSVMHEALHNHYTQKSLSLSPGK*
hIgG4 Heavy Chain Constant Region
(SEQ ID NO: 13)
ASTKGPSVFPLAPCSRSTSESTAALGCLVKDYFPEPVTVSWNSGALTSG
VHTFPAVLQSSGLYSLSSVVTVPSSSLGTKTYTCNVDHKPSNTKVDKRV
ESKYGPPCPSCPAPEFLGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDK
SRWQEGNVFSCSVMHEALHNHYTQKSLSLSPGK*
hIgG4 Heavy Chain Constant Region
(SEQ ID NO: 20)
ASTKGPSVFPLAPCSRSTSESTAALGCLVKDYFPEPVTVSWNSGALTSG
VHTFPAVLQSSGLYSLSSVVTVPSSSLGTKTYTCNVDHKPSNTKVDKRV
ESKYGPPCPSCPAPEFLGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDK
SRWQEGNVFSCSVMHEALHNHYTQKSLSLSLGK*
hIgG4 mut Heavy Chain Constant Region
(SEQ ID NO: 14)
ASTKGPSVFPLAPCSRSTSESTAALGCLVKDYFPEPVTVSWNSGALTSG
VHTFPAVLQSSGLYSLSSVVTVPSSSLGTKTYTCNVDHKPSNTKVDKRV
ESKYGPPCPPCPAPEFLGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDK
SRWQEGNVFSCSVMHEALHNHYTQKSLSLSPGK*
hIgG4 mut Heavy Chain Constant Region
(SEQ ID NO: 21)
ASTKGPSVFPLAPCSRSTSESTAALGCLVKDYFPEPVTVSWNSGALTSG
VHTFPAVLQSSGLYSLSSVVTVPSSSLGTKTYTCNVDHKPSNTKVDKRV
ESKYGPPCPPCPAPEFLGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDK
SRWQEGNVFSCSVMHEALHNHYTQKSLSLSLGK*

The term “G9.2-17(Ig4)” used herein refers to a G9.2-17 antibody which is an IgG4 molecule. Likewise, the term “G9.2-17 (Fab)” refers to a G9.2-17 antibody, which is a Fab molecule. The heavy and light chain CDRs of reference antibody G9.2-17 are provided in Table 1 (determined using the Kabat methodology).

In some embodiments, anti-Galectin antibodies having any of the above heavy chain constant regions are paired with a light chain having the following light chain constant region:

Light Chain Constant Region
(SEQ ID NO: 11)
TVAAPSVFIFPPSDEQLKSGTASVVCLLNNFYPREAKVQNKVDNALQSG
NSQESVTEQDSKDSTYSLSSTLTLSKADYEKHKVYACEVTHQGLSSPVT
KSFNRGEC

Exemplary full length anti-Galectin-9 antibodies are provided below:

G9.2-17 hIgG1 Heavy Chain
(SEQ ID NO: 16)
EVQLVESGGGLVQPGGSLRLSCAASGETVSSSSIHWVRQAPGKGLEWVAY
ISSSSGYTYYADSVKGRFTISADTSKNTAYLQMNSLRAEDTAVYYCARYW
SYPSWWPYRGMDYWGQGTLVTVSSASTKGPSVFPLAPSSKSTSGGTAALG
CLVKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVFLF
PPKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQP
REPQVYTLPPSREEMTKNQVSLTCLVKGFYPSDIAVEWESNGQPENNYKT
TPPVLDSDGSFFLYSKLTVDKSRWQQGNVESCSVMHEALHNHYTQKSLSL
SPGK*
G9.2-17 hIgG1 LALA Heavy Chain
(SEQ ID NO: 17)
EVQLVESGGGLVQPGGSLRLSCAASGETVSSSSIHWVRQAPGKGLEWVAY
ISSSSGYTYYADSVKGRFTISADTSKNTAYLQMNSLRAEDTAVYYCARYW
SYPSWWPYRGMDYWGQGTLVTVSSASTKGPSVFPLAPSSKSTSGGTAALG
CLVKDYFPEPVTVSWNSGALTSGVHTFPAVLQSSGLYSLSSVVTVPSSSL
GTQTYICNVNHKPSNTKVDKKVEPKSCDKTHTCPPCPAPEAAGGPSVFLF
PPKPKDTLMISRTPEVTCVVVDVSHEDPEVKFNWYVDGVEVHNAKTKPRE
EQYNSTYRVVSVLTVLHQDWLNGKEYKCKVSNKALGAPIEKTISKAKGQP
REPQVYTLPPSREEMTKNQVSLTCLVKGFYPSDIAVEWESNGQPENNYKT
TPPVLDSDGSFFLYSKLTVDKSRWQQGNVESCSVMHEALHNHYTQKSLSL
SPGK*
G9.2-17 hIgG4 Heavy Chain
(SEQ ID NO: 18)
EVQLVESGGGLVQPGGSLRLSCAASGETVSSSSIHWVRQAPGKGLEWVAY
ISSSSGYTYYADSVKGRFTISADTSKNTAYLQMNSLRAEDTAVYYCARYW
SYPSWWPYRGMDYWGQGTLVTVSSASTKGPSVFPLAPCSRSTSESTAALG
CLVKDYFPEPVTVSWNSGALTSGVHTFPAVLQSSGLYSLSSVVTVPSSSL
GTKTYTCNVDHKPSNTKVDKRVESKYGPPCPSCPAPEFLGGPSVFLFPPK
PKDTLMISRTPEVTCVVVDVSQEDPEVQFNWYVDGVEVHNAKTKPREEQF
NSTYRVVSVLTVLHQDWLNGKEYKCKVSNKGLPSSIEKTISKAKGQPREP
QVYTLPPSQEEMTKNQVSLTCLVKGFYPSDIAVEWESNGQPENNYKTTPP
VLDSDGSFFLYSRLTVDKSRWQEGNVESCSVMHEALHNHYTQKSLSLSPG
K*
G9.2-17 hIgG4 Heavy Chain
(SEQ ID NO: 22)
EVQLVESGGGLVQPGGSLRLSCAASGETVSSSSIHWVRQAPGKGLEWVAY
ISSSSGYTYYADSVKGRFTISADTSKNTAYLQMNSLRAEDTAVYYCARYW
SYPSWWPYRGMDYWGQGTLVTVSSASTKGPSVFPLAPCSRSTSESTAALG
CLVKDYFPEPVTVSWNSGALTSGVHTFPAVLQSSGLYSLSSVVTVPSSSL
GTKTYTCNVDHKPSNTKVDKRVESKYGPPCPSCPAPEFLGGPSVFLFPPK
PKDTLMISRTPEVTCVVVDVSQEDPEVQFNWYVDGVEVHNAKTKPREEQF
NSTYRVVSVLTVLHQDWLNGKEYKCKVSNKGLPSSIEKTISKAKGQPREP
QVYTLPPSQEEMTKNQVSLTCLVKGFYPSDIAVEWESNGQPENNYKTTPP
VLDSDGSFFLYSRLTVDKSRWQEGNVESCSVMHEALHNHYTQKSLSLSLG
K*
G9.2-17 hIgG4 Fab Arm Exchange mut Heavy Chain
(SEQ ID NO: 19)
EVQLVESGGGLVQPGGSLRLSCAASGFTVSSSSIHWVRQAPGKGLEWVAY
ISSSSGYTYYADSVKGRFTISADTSKNTAYLQMNSLRAEDTAVYYCARYW
SYPSWWPYRGMDYWGQGTLVTVSSASTKGPSVFPLAPCSRSTSESTAALG
CLVKDYFPEPVTVSWNSGALTSGVHTFPAVLQSSGLYSLSSVVTVPSSSL
GTKTYTCNVDHKPSNTKVDKRVESKYGPPCPPCPAPEFLGGPSVFLFPPK
PKDTLMISRTPEVTCVVVDVSQEDPEVQFNWYVDGVEVHNAKTKPREEQF
NSTYRVVSVLTVLHQDWLNGKEYKCKVSNKGLPSSIEKTISKAKGQPREP
QVYTLPPSQEEMTKNQVSLTCLVKGFYPSDIAVEWESNGQPENNYKTTPP
VLDSDGSFFLYSRLTVDKSRWQEGNVESCSVMHEALHNHYTQKSLSLSPG
K*
G9.2-17 hIgG4 Fab Arm Exchange mut Heavy Chain
(SEQ ID NO: 23)
EVQLVESGGGLVQPGGSLRLSCAASGFTVSSSSIHWVRQAPGKGLEWVAY
ISSSSGYTYYADSVKGRFTISADTSKNTAYLQMNSLRAEDTAVYYCARYW
SYPSWWPYRGMDYWGQGTLVTVSSASTKGPSVFPLAPCSRSTSESTAALG
CLVKDYFPEPVTVSWNSGALTSGVHTFPAVLQSSGLYSLSSVVTVPSSSL
GTKTYTCNVDHKPSNTKVDKRVESKYGPPCPPCPAPEFLGGPSVFLFPPK
PKDTLMISRTPEVTCVVVDVSQEDPEVQFNWYVDGVEVHNAKTKPREEQF
NSTYRVVSVLTVLHQDWLNGKEYKCKVSNKGLPSSIEKTISKAKGQPREP
QVYTLPPSQEEMTKNQVSLTCLVKGFYPSDIAVEWESNGQPENNYKTTPP
VLDSDGSFFLYSRLTVDKSRWQEGNVESCSVMHEALHNHYTQKSLSLSLG
K*

Any of the above heavy chain can be paired with a Light Chain of (SEQ ID NO: 15) shown below:

DIQMTQSPSSLSASVGDRVTITCRASQSVSSAVAWYQQKPGKAPKLLIYS
ASSLYSGVPSRFSGSRSGTDFTLTISSLQPEDFATYYCQQSSTDPITFGQ
GTKVEIKRTVAAPSVFIFPPSDEQLKSGTASVVCLLNNFYPREAKVQNKV
DNALQSGNSQESVTEQDSKDSTYSLSSTLTLSKADYEKHKVYACEVTHQG
LSSPVTKSFNRGEC*

Any of the anti-Galectin 9 antibodies disclosed herein may be produced by any method known in the art, including but not limited to, recombinant technology. The anti-Galectin-9 antibodies thus prepared can be characterized using methods known in the art, whereby reduction, amelioration, or neutralization of Galectin-9 biological activity is detected and/or measured. For example, in some embodiments, an ELISA-type assay is suitable for qualitative or quantitative measurement of Galectin-9 inhibition of Dectin-1 or TIM-3 signaling.

The bioactivity of an anti-Galectin-9 antibody can verified by incubating a candidate antibody with Galectin-9, and monitoring any one or more of the following characteristics: (a) binding between Dectin-1 and Galectin-9 and inhibition of the signaling transduction mediated by the binding; (b) preventing, ameliorating, or treating any aspect of a solid tumor as those described herein; (c) blocking or decreasing Dectin-1 activation; (d) inhibiting (reducing) synthesis, production or release of Galectin-9. Alternatively, TIM-3 can be used to verify the bioactivity of an anti-Galectin-9 antibody using the protocol described above. Alternatively, CD206 can be used to verify the bioactivity of an anti-Galectin-9 antibody using the protocol described above.

In some embodiments, bioactivity or efficacy is assessed in a subject, e.g., by measuring peripheral and intra-tumoral T cell ratios, T cell activation, or by macrophage phenotyping. Additional assays to determine bioactivity of an anti-Galectin-9 antibody include measurement of CD8+ and CD4+ (conventional) T-cell activation (in an in vitro or in vivo assay, e.g., by measuring inflammatory cytokine levels, e.g., IFNgamma, TNFalpha, CD44, ICOS granzymeB, Perforin, IL2 (upregulation); CD26L and IL-10 (downregulation)); measurement of reprogramming of macrophages (in vitro or in vivo), e.g., from the M2 to the M1 phenotype (e.g., increased MHCII, reduced CD206, increased TNF-alpha and iNOS), Alternatively, levels of ADCC can be assessed, e.g., in an in vitro assay, as described herein.

The anti-Galectin-9 antibodies, as well as the encoding nucleic acids or nucleic acid sets, vectors comprising such, as described herein can be mixed with a pharmaceutically acceptable carrier (excipient) to form a pharmaceutical composition for use in treating a target disease. “Acceptable” means that the carrier must be compatible with the active ingredient of the composition (and preferably, capable of stabilizing the active ingredient) and not deleterious to the subject to be treated. Pharmaceutically acceptable excipients (carriers) including buffers, which are well known in the art. See, e.g., Remington: The Science and Practice of Pharmacy 20th Ed. (2000) Lippincott Areiams and Wilkins, Ed. K. E. Hoover.

The pharmaceutical compositions to be used in the present methods can comprise pharmaceutically acceptable carriers, excipients, or stabilizers in the form of lyophilized formulations or aqueous solutions. (Remington: The Science and Practice of Pharmacy 20th Ed. (2000) Lippincott Areiams and Wilkins, Ed. K. E. Hoover). Acceptable carriers, excipients, or stabilizers are nontoxic to recipients at the dosages and concentrations used, and comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). In some examples, the pharmaceutical composition described herein comprises liposomes containing the antibodies (or the encoding nucleic acids) which can be prepared by methods known in the art, such as described in Epstein, et al., Proc. Natl. Acad. Sci. USA 82:3688 (1985); Hwang, et al., Proc. Natl. Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

In some embodiments, the anti-Galectin-9 antibodies, or the encoding nucleic acid(s), are be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are known in the art, see, e.g., Remington, The Science and Practice of Pharmacy 20th Ed. Mack Publishing (2000).

In other examples, the pharmaceutical composition described herein can be formulated in sustained-release format. Suitable examples of sustained-release preparations include semipermeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinyl alcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(-)-3-hydroxybutyric acid.

The pharmaceutical compositions to be used for in vivo administration must be sterile. This is readily accomplished by, for example, filtration through sterile filtration membranes. Therapeutic antibody compositions are generally placed into a container having a sterile access port, for example, an intravenous solution bag or vial having a stopper pierceable by a hypodermic injection needle.

The pharmaceutical compositions described herein can be in unit dosage forms such as tablets, pills, capsules, powders, granules, solutions or suspensions, or suppositories, for oral, parenteral or rectal administration, or administration by inhalation or insufflation.

For preparing solid compositions such as tablets, the principal active ingredient can be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate. Suitable surface-active agents include, in particular, non-ionic agents, such as polyoxyethylenesorbitans (e.g., Tween™ 20, 40, 60, 80 or 85) and other sorbitans (e.g., Span™ 20, 40, 60, 80 or 85). Compositions with a surface-active agent are conveniently comprise between 0.05 and 5% surface-active agent, and can be between 0.1 and 2.5%. It are be appreciated that other ingredients may be added, for example mannitol or other pharmaceutically acceptable vehicles, if necessary.

Suitable emulsions may be prepared using commercially available fat emulsions, such as Intralipid™, Liposyn™, Infonutrol™, Lipofundin™ and Lipiphysan™. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g. egg phospholipids, soybean phospholipids or soybean lecithin) and water. It are be appreciated that other ingredients may be added, for example glycerol or glucose, to adjust the tonicity of the emulsion. Suitable emulsions are typically contain up to 20% oil, for example, between 5 and 20%. The fat emulsion can comprise fat droplets between 0.1 and 1.0 .im, particularly 0.1 and 0.5 .im, and have a pH in the range of 5.5 to 8.0.

The emulsion compositions can be those prepared by mixing an antibody with Intralipid™ or the components thereof (soybean oil, egg phospholipids, glycerol and water).

Pharmaceutical compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect.

Compositions in preferably sterile pharmaceutically acceptable solvents may be nebulized by use of gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

B. Treatment with Anti-Galectin-9 Antibodies

Any of the anti-Galectin 9 antibodies or a pharmaceutical composition comprising such may be used for treating a cancer patient diagnosed by the methods disclosed herein.

To perform the treatment methods disclosed herein, an effective amount of any of the anti-Galectin 9 antibodies as disclosed (e.g., G9.2-17 or a functional equivalent thereof) herein may be administered to the subject. In some embodiments, the anti-Galectin-9 antibody is G9.2-17. In some embodiments, the anti-Galectin-9 antibody is an antibody having the same heavy chain CDR sequences and/or the same light chain CDR sequences as reference antibody G9.2-17. In some embodiments, the anti-Galectin-9 antibody is an antibody having the same VH and VL sequences as reference antibody G9.2-17. In some embodiments, such an antibody is an IgG1 molecule (e.g., having a wild-type IgG1 constant region or a mutant thereof as those disclosed herein). Alternatively, the antibody is an IgG4 molecule (e.g., having a wild-type IgG4 constant region or a mutant thereof as those described herein). In some embodiments, the antibody comprises a light chain complementarity determining region 1 (CDR1) set forth as SEQ ID NO: 1, a light chain complementary determining region 2 (CDR2) set forth as SEQ ID NO: 2, and a light chain complementary determining region 3 (CDR3) set forth as SEQ ID NO: 3 and/or comprises a heavy chain complementarity determining region 1 (CDR1) set forth as SEQ ID NO: 4, a heavy chain complementary determining region 2 (CDR2) set forth as SEQ ID NO: 5, and a heavy chain complementary determining region 3 (CDR3) set forth as SEQ ID NO: 6. In some embodiments, the antibody comprises a heavy chain variable region comprising SEQ ID NO: 7. In some embodiments, the antibody comprises a light chain variable region comprising SEQ ID NO: 8. In some embodiments, the antibody comprises a heavy chain variable region comprising SEQ ID NO: 7. In some embodiments, the antibody comprises a light chain variable region comprising SEQ ID NO: 8 and heavy chain comprising SEQ ID NO: 19. In some embodiments, the antibody comprises a light chain comprising SEQ ID NO: 15. In specific examples, the anti-Galectin-9 antibody used herein has a heavy chain of SEQ ID NO:19 and a light chain of SEQ ID NO:15. In some embodiments, the antibody is G9.2-17 IgG4.

An effective amount of the pharmaceutical composition described herein can be administered to a subject (e.g., a human) in need of the treatment via a suitable route, systemically or locally. In some embodiments, the anti-Galectin-9 antibodies are administered by intravenous administration, e.g., as a bolus or by continuous infusion over a period of time, by intramuscular, intraperitoneal, intracerebrospinal, subcutaneous, intra-arterial, intra-articular, intrasynovial, intrathecal, intratumoral, oral, inhalation or topical routes. In one embodiment, the anti-Galectin-9 antibody is administered to the subject by intravenous infusion. Commercially available nebulizers for liquid formulations, including jet nebulizers and ultrasonic nebulizers are useful for administration. Liquid formulations can be directly nebulized and lyophilized powder can be nebulized after reconstitution. Alternatively, the antibodies as described herein can be aerosolized using a fluorocarbon formulation and a metered dose inhaler, or inhaled as a lyophilized and milled powder.

As used herein, “an effective amount” refers to the amount of each active agent required to confer therapeutic effect on the subject, either alone or in combination with one or more other active agents. In some embodiments, the therapeutic effect is reduced Galectin-9 activity and/or amount/expression, reduced Dectin-1 signaling, reduced TIM-3 signaling, reduced CD206 signaling, or increased anti-tumor immune responses in the tumor microenvironment. Non-limiting examples of increased anti-tumor responses include increased activation levels of effector T cells, or switching of the TAMs from the M2 to the M1 phenotype. In some cases, the anti-tumor response includes increased ADCC responses. Determination of whether an amount of the antibody achieved the therapeutic effect would be evident to one of skill in the art. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment.

Empirical considerations, such as the half-life, generally contribute to the determination of the dosage. For example, antibodies that are compatible with the human immune system, such as humanized antibodies or fully human antibodies, are in some instances used to prolong half-life of the antibody and to prevent the antibody being attacked by the host's immune system. Frequency of administration may be determined and adjusted over the course of therapy, and is generally, but not necessarily, based on treatment and/or suppression and/or amelioration and/or delay of a target disease/disorder. Alternatively, sustained continuous release formulations of an antibody may be appropriate. Various formulations and devices for achieving sustained release are known in the art.

In one example, dosages for an antibody as described herein are determined empirically in individuals who have been given one or more administration(s) of the antibody. Individuals are given incremental dosages of the antagonist. To assess efficacy of the antagonist, an indicator of the disease/disorder can be followed.

In some embodiments, the methods of the present disclosure increase anti-tumor activity (e.g., reduce cell proliferation, tumor growth, tumor volume, and/or tumor burden or load or reduce the number of metastatic lesions over time) by at least about 10%, 20%, 25%, 30%, 40%, 50%, 60%, 70%, 75%, 80%, 85%, 90%, 95%, or more as compared to levels prior to treatment or in a control subject. In some embodiments, reduction is measured by comparing cell proliferation, tumor growth, and/or tumor volume in a subject before and after administration of the pharmaceutical composition. In some embodiments, the method of treating or ameliorating a cancer in a subject allows one or more symptoms of the cancer to improve by at least about 10%, 20%, 30%, 40%, 50%, 60%, 70%, 80%, 90%, 95%, or more. In some embodiments, before, during, and after the administration of the pharmaceutical composition, cancerous cells and/or biomarkers in a subject are measured in a biological sample, such as blood, serum, plasma, urine, peritoneal fluid, and/or a biopsy from a tissue or organ. In some embodiments, the methods include administration of the compositions of the invention to reduce tumor volume, size, load or burden in a subject to an undetectable size, or to less than about 1%, 2%, 5%, 10%, 20%, 25%, 30%, 40%, 50%, 60%, 70%, 75%, 80%, or 90% of the subject's tumor volume, size, load or burden prior to treatment. In other embodiments, the methods include administration of the compositions of the invention to reduce the cell proliferation rate or tumor growth rate in a subject to an undetectable rate, or to less than about 1%, 2%, 5%, 10%, 20%, 25%, 30%, 40%, 50%, 60%, 70%, 75%, 80%, or 90% of the rate prior to treatment. In other embodiments, the methods include administration of the compositions of the invention to reduce the development of or the number or size of metastatic lesions in a subject to an undetectable rate, or to less than about 1%, 2%, 5%, 10%, 20%, 25%, 30%, 40%, 50%, 60%, 70%, 75%, 80%, or 90% of the rate prior to treatment.

As used herein, the term “treating” refers to the application or administration of a composition including one or more active agents to a subject, who has a target disease or disorder, a symptom of the disease/disorder, or a predisposition toward the disease/disorder, with the purpose to cure, heal, alleviate, relieve, alter, remedy, ameliorate, improve, or affect the disorder, a symptom of the disease or disorder, or the predisposition toward the disease or disorder.

Alleviating a target disease/disorder includes delaying the development or progression of the disease, or reducing disease severity or prolonging survival. Alleviating the disease or prolonging survival does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a target disease or disorder includes initial onset and/or recurrence.

Conventional methods, known to those of ordinary skill in the art of medicine, can be used to administer the pharmaceutical composition to the subject, depending upon the type of disease to be treated or the site of the disease. In some embodiments, the anti-Galectin-9 antibody can be administered to a subject by intravenous infusion.

Injectable compositions may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous injection, water soluble antibodies can be administered by the drip method, whereby a pharmaceutical formulation containing the antibody and a physiologically acceptable excipient is infused. Physiologically acceptable excipients may include, for example, 5% dextrose, 0.9% saline, Ringer's solution or other suitable excipients. Intramuscular preparations, e.g., a sterile formulation of a suitable soluble salt form of the antibody, can be dissolved and administered in a pharmaceutical excipient such as Water-for-Injection, 0.9% saline, or 5% glucose solution.

In some embodiments, the anti-Galectin-9 antibodies described herein are be used as a monotherapy for treating the target cancer disclosed herein, i.e., free of other anti-cancer therapy concurrently with the therapy using the anti-Galectin-9 antibody.

In other embodiments, the treatment method further comprises administering to the subject an inhibitor of a checkpoint molecule, for example, PD-1. Examples of PD-1 inhibitors include anti-PD-1 antibodies, such as pembrolizumab, nivolumab, and cemiplimab. Such checkpoint inhibitors can be administered simultaneously or sequentially (in any order) with the anti-Galectin-9 antibody according to the present disclosure. In some embodiments, the checkpoint molecule is PD-L1. Examples of PD-L1 inhibitors include anti-PD-L1 antibodies, such as durvalumab, avelumab, and atezolizumab. In some embodiments, the checkpoint molecule is CTLA-4. An example of a CTLA-4 inhibitor is the anti-CTLA-4 antibody ipilimumab. In some embodiments, the inhibitor targets a checkpoint molecule selected from CD40, GITR, LAG-3, OX40, TIGIT and TIM-3.

In some embodiments, the methods are provided, the anti-Galectin-9 antibody is administered concurrently with a checkpoint inhibitor. In some embodiments, the anti-Galectin-9 antibody is administered before or after a checkpoint inhibitor. In some embodiments, the checkpoint inhibitor is administered systemically. In some embodiments, the checkpoint inhibitor is administered locally. In some embodiments, the checkpoint inhibitor is administered by intravenous administration, e.g., as a bolus or by continuous infusion over a period of time, by intramuscular, intraperitoneal, intracerebrospinal, subcutaneous, intra-arterial, intra-articular, intrasynovial, intrathecal, intratumoral, oral, inhalation or topical routes. In one embodiment, the checkpoint inhibitor is administered to the subject by intravenous infusion.

### III. Kit for Cancer Diagnosis

The present disclosure also provides kits for use in any of the diagnostic methods disclosed herein, optionally for use in any of the treatment methods also disclosed herein.

In some embodiments, the kits disclosed herein comprise one or more agents for measuring levels of galectin-9 in biological samples. Such agents can be antibodies specific to galectin-9. Alternatively, the agents may be nucleic acids for measuring mRNA levels of galectin-9 in a biological sample. The kit may further comprise reagents or devices for collecting and processing biological samples, and optionally containers for placing the biological samples. In some examples, the kit may further comprise one or more therapeutic agents for treating breast cancer, for example, any of the anti-Galectin 9 antibodies as disclosed herein.

Any of the kits disclosed herein may comprise one or more containers for placing the one or more detection agents and optionally reagents and/or therapeutic agents. In some embodiments, the kit can comprise instructions for use in accordance with any of the methods described herein. The included instructions can comprise a description of collecting biological samples, processing such, and measuring Galectin-9 levels in such biological samples. In addition, the included instructions may further comprise descriptions for identifying breast cancer patients, determining their tumor burden and/or tumor status, disease progression levels, responsiveness to a currently treatment, and/or potential survival rates according to any of the methods disclosed herein. Further, the instructions may comprise descriptions of selecting suitable treatment and how to apply such a treatment to the patient. Instructions supplied in the kits disclosed herein are typically written instructions on a label or package insert (e.g., a paper sheet included in the kit), but machine-readable instructions (e.g., instructions carried on a magnetic or optical storage disk) are also acceptable.

The kits disclosed herein are in suitable packaging. Suitable packaging includes, but is not limited to, vials, bottles, jars, flexible packaging (e.g., sealed Mylar or plastic bags), and the like. Any of the kits may optionally provide additional components such as buffers and interpretive information. Normally, the kit comprises a container and a label or package insert(s) on or associated with the container. In some embodiments, the present disclosure provides articles of manufacture comprising contents of the kits described above.

General Techniques

The practice of the present invention are employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook, et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel, et al., eds., 1987); PCR: The Polymerase Chain Reaction, (Mullis, et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1. Assessment of Galectin-9 Expression in Tumor Biopsy-derived Organoid Fractions

Biopsy derived organoids can be used as a proxy to assess levels of Galectin-9 in the original tumor. Accordingly, the ability to assess Galectin-9 levels in single cell or organoid fractions was tested.

Biopsies were received from representative pancreatic adenocarcinoma and colorectal cancers and processed as follows. Human surgically resected tumor specimens were received fresh in DMEM media on ice and minced in 10 cm dishes. Minced tumors were resuspended in DMEM+10% FBS with 100 U/mL collagenase type IV to obtain spheroids. Partially digested samples were pelleted and then re-suspended in fresh DMEM+10% FBS and strained over both 100 mm and 40 mm filters to generate S1 (>100 mm), S2 (40-100 mm), and S3 (<40 mm) spheroid fractions, which were subsequently maintained in ultra-low-attachment tissue culture plates.

S2 fractions were digested by trypsin for 15 mins to generate into single cells. For flow cytometry preparation, cell pellets from S2 and S3 fractions were re-suspended and cell labeling was performed after Fc receptor blocking (#422301; BioLegend, San Diego, Calif.) by incubating cells with fluorescently conjugated mAbs directed against human CD45 (HI30), CD3 (UCHT1), CD11b (M1/70), Epcam (9C4) and Gal9 (9M1-3; all Biolegend) or Gal9 Fab of G9.2-17 or Fab isotype. Dead cells were excluded from analysis using zombie yellow (BioLegend). Flow cytometry was carried out on the Attune NxT flow cytometer (Thermo Scientific). Data were analyzed using FlowJo v.10.1 (Treestar, Ashland, Oreg.).

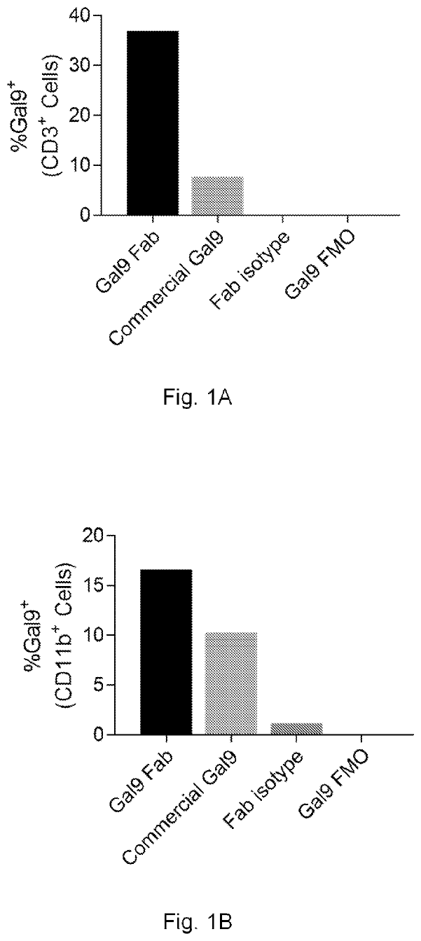

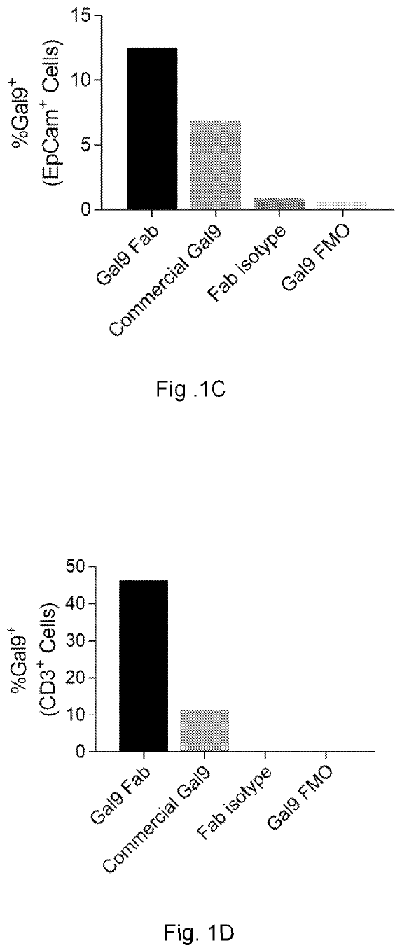

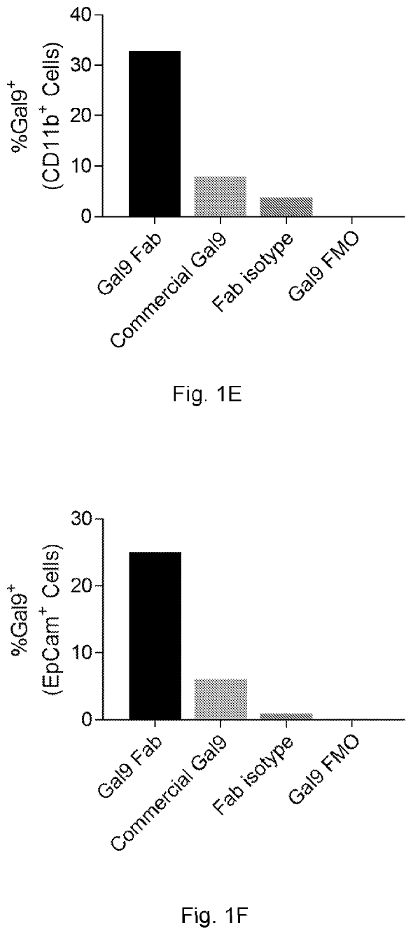

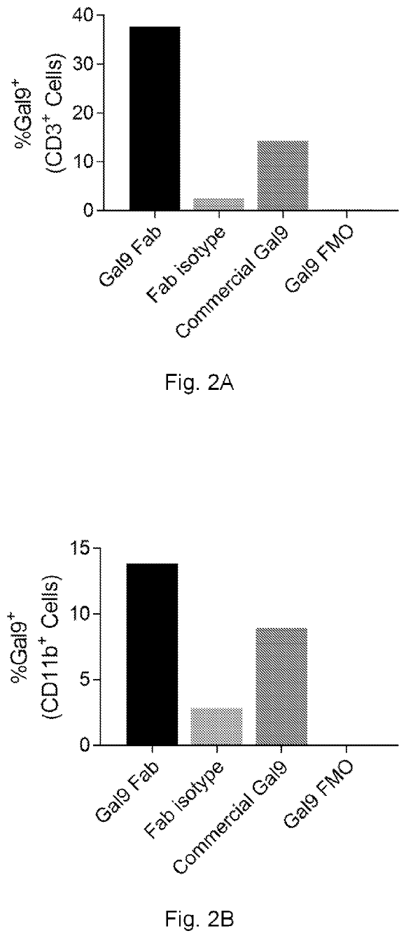

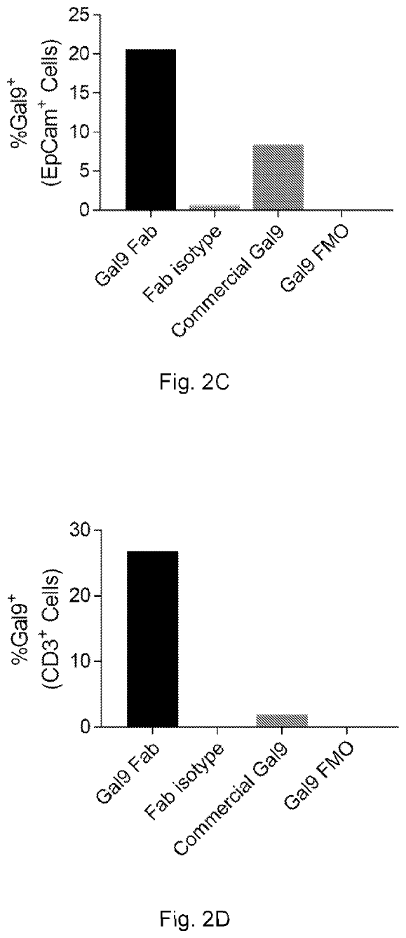

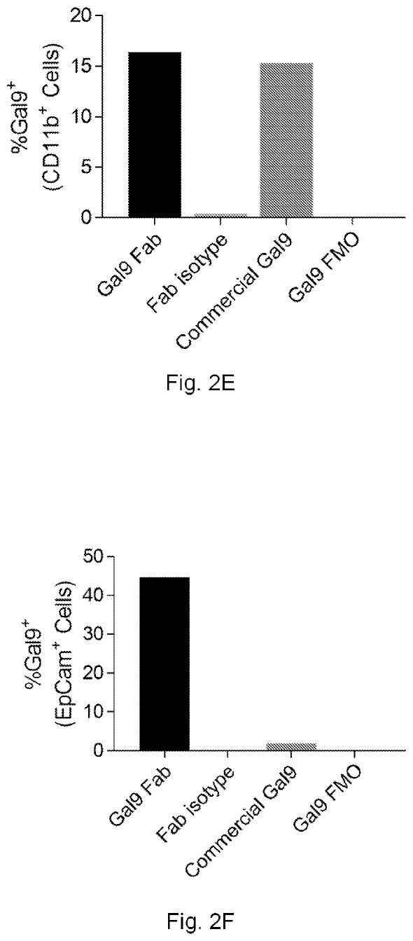

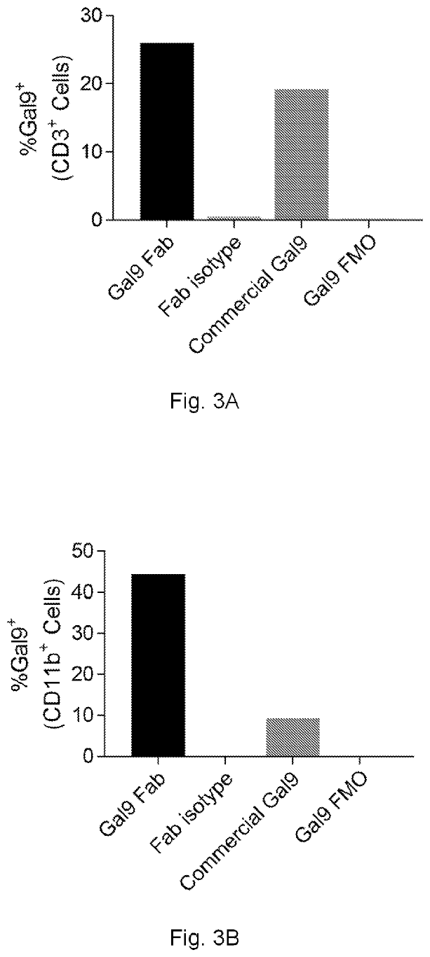

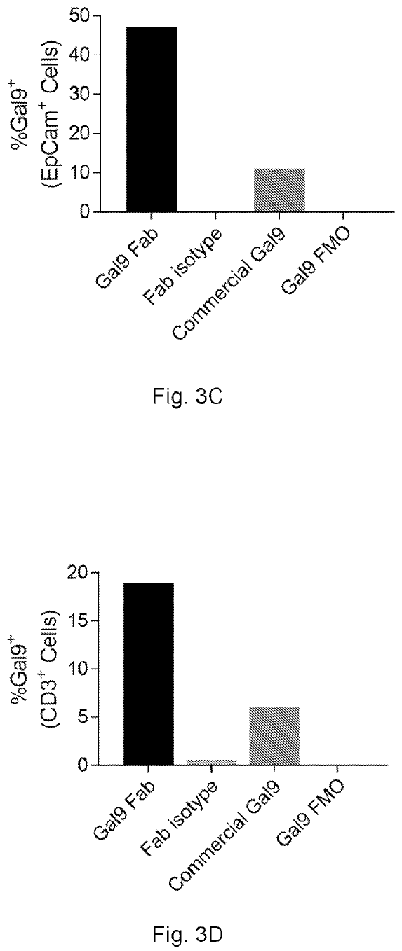

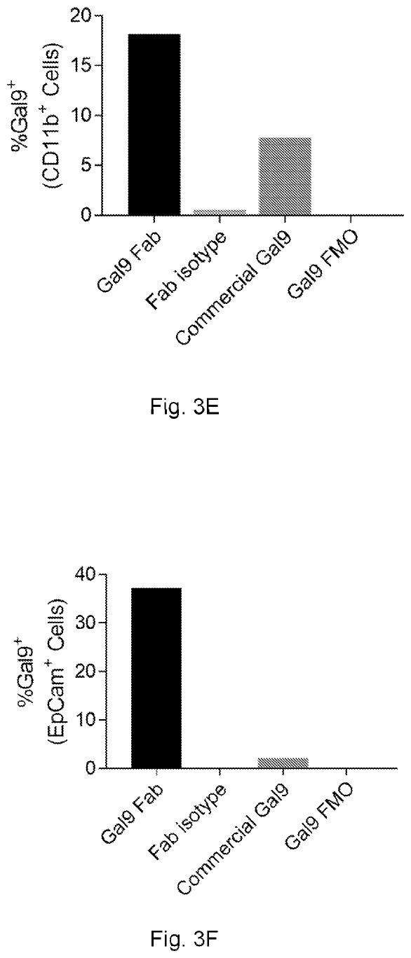

Results are shown in FIGS. 1A-1F, 2A-2F and 3A-3F and indicate that levels of Galectin-9 detected by the Gal9 G9.2-17 Fab in S2 single cell and S3 organoid fractions correlate. Accordingly, both S2 single cells and S3 organoids can be used for assessment of Galectin-9 levels in organoids derived from tumor biopsies.

Additionally, pancreatic adenocarcinoma (PDA), colorectal cancer (CRC), and hepatocellular carcinoma (HCC) tumors were processed as described above. The table below indicates that both S2 single cells and S3 organoids can be used for assessment of galectin-9 levels in organoids derived from tumor biopsies.

TABLE 4
Gal9 expression on EPCAM + tumor cells

| | S2 Single Cell Fractions | | S3 Organoid Fractions | |
|---|---|---|---|---|
| | % Gal9 | % Gal9 | % Gal9 | % Gal9 |
| | expression based | expression based | expression based | expression based |
| | on Gal9 Fab of | on Gal9 Fab | on Gal9 Fab of | on Gal9 Fab |
| Tumor type | G9.2-17 staining | isotype staining | G9.2-17 staining | isotype staining |
| PDA | 12.5 | 0.88 | 25 | 0.94 |
| CRC liver | 33.5 | 0.15 | 42.2 | 0.06 |
| metastasis | | | | |
| Liver NET | 13.8 | 0.45 | 64.3 | 0.02 |
| CRC | 20.6 | 0.64 | 44.7 | 0.21 |
| PDA | 47 | 0.15 | 37.2 | 0.17 |
| Pancreas NET | 15.8 | 2.41 | 12.3 | 4.19 |
| CRC | 9.15 | 3.97 | 2.9 | 2.27 |
| CRC | | | 4.58 | 0.55 |
| HCC | | | 49.8 | 5.84 |

Galectin-9 acts as a potent mediator of cancer-associated immunosuppression and is expressed on tumor-associated macrophages, as well as intra-tumoral immunosuppressive gamma delta T cells. Table 5 shows galectin-9 expression on macrophages as seen in both S2 single cells and S3 organoids and Table 6 shows galectin-9 expression on T cells as seen in both S2 single cells and S3 organoids. Table 7 shows expression of the delta 1 chain of a T cell receptor in S2 single cells and S3 organoids as detected by a delta 1 Fab and Fab isotype.

TABLE 5
Gal9 expression on CD11b + myeloid cells

| | S2 Single Cell Fractions | | S3 Organoid Fractions | |
|---|---|---|---|---|
| | % Gal9 | % Gal9 | % Gal9 | % Gal9 |
| | expression based | expression based | expression based | expression based |
| | on Gal9 Fab of | on Gal9 Fab | on Gal9 Fab of | on Gal9 Fab |
| Tumor type | G9.2-17 staining | isotype staining | G9.2-17 staining | isotype staining |
| PDA | 16.6 | 1.19 | 32.8 | 3.83 |
| CRC liver | 45.9 | 1.2 | 49.8 | 1.5 |
| metastasis | | | | |
| Liver NET | | | | |
| CRC | 13.8 | 2.86 | 16.4 | 0.38 |
| PDA | 44.4 | 0 | 18.2 | 0.54 |
| Pancreas NET | 45.2 | 16.2 | 34.3 | 1.87 |
| CRC | 15 | 5.84 | 15.6 | 11.8 |
| CRC | | | 6.16 | 2.4 |
| HCC | | | 26.2 | 1.72 |

TABLE 6
Gal9 expression on CD3 + T cells

| | S2 Single Cell Fractions | | S3 Organoid Fractions | |
|---|---|---|---|---|
| | % Gal9 | % Gal9 | % Gal9 | % Gal9 |
| | expression based | expression based | expression based | expression based |
| | on Gal9 Fab of | on Gal9 Fab | on Gal9 Fab of | on Gal9 Fab |
| Tumor type | G9.2-17 staining | isotype staining | G9.2-17 staining | isotype staining |
| PDA | 36.9 | 0 | 46.3 | 0 |
| CRC liver | 31.1 | 0.56 | 34.2 | 0 |
| metastasis | | | | |
| Liver NET | | | | |
| CRC | 37.7 | 2.5 | 26.8 | 0 |
| PDA | 26 | 0.52 | 18.9 | 0.59 |
| Pancreas NET | 63.7 | 32.2 | 27.5 | 4.96 |
| CRC | 12.5 | 1.59 | 25 | 15.3 |
| CRC | | | 3.74 | 0.87 |
| HCC | | | 12.3 | 1.14 |
| Liver NET | 3.32 | 0.27 | 50.4 | 25.3 |
| CRC | 19.9 | 0.93 | 41.3 | 1.61 |
| PDA | 41.3 | 55 | 46.9 | 1.49 |
| PDA | 24.7 | 4.49 | 11.1 | 3.21 |
| Pancreas NET | 23 | 39.1 | 5.77 | 2.61 |
| CRC | 36.5 | 5.08 | 13.1 | 3.54 |
| CRC | | | 17.5 | 0.85 |
| CRC | | | 55.2 | 0.33 |
| CRC liver | | | 22.8 | 0.6 |
| metastasis | | | | |
| CRC | | | 33 | 0 |
| HCC | | | 35.1 | 0.3 |

The above results show that high levels of Galectin-9 were detected in patient blood/tissue samples compared with healthy controls. Analyses of PDOTs show high levels of Galectin-9 on tumor, myeloid and T cells, suggesting Galectin-9 as a promising disease specific target. Additionally, measurement of Galectin-9 levels in a tumor may constitute a means for determining a population of cancer patients that are likely to respond to anti-Galectin-9 therapy.

### Example 2. Preparation of Patient-Derived Organotypic Tumor Spheroids (PDOTs) for Cellular Analysis

Biopsy-derived organoids can be a useful measure to assess the ability of a therapeutic to stimulate an immune response. Accordingly, S2 fractions described in the previous Example 1 above used for ex vivo culture were treated with anti-Galectin-9 antibody G9.2-17 and prepared for immune profiling.

An aliquot of the S2 fraction was pelleted and resuspended in type I rat tail collagen (Corning) at a concentration of 2.5 mg/mL following the addition of 10×PBS with phenol red with pH adjusted using NaOH. pH 7.0-7.5 is confirmed using PANPEHA Whatman paper (Sigma-Aldrich). The spheroid-collagen mixture is then injected into the center gel region of a 3-D microfluidic culture device as described in Jenkins et al., Cancer Discov. 2018 February; 8(2):196-215; Ex Vivo Profiling of PD-1 Blockade Using Organotypic Tumor Spheroids, the contents of which is herein incorporated by reference in its entirety. Collagen hydrogels containing patient-derived organotypic tumor spheroids (PDOTS) were hydrated with media with or without anti-Galectin-9 monoclonal antibody G9.2-17 after 30 minutes at 37° C. The PDOTS were then incubated at 37° C. for 3 days.

Cell pellets were re-suspended in the FACS buffer and 1×10⁶ cells were first stained with zombie yellow (BioLegend) to exclude dead cells. After viability staining, cells were incubated with an anti-CD16/CD32 mAb (eBiosciences, San Diego, Calif.) for blocking FcγRIII/II followed by antibody staining with 1 μg of fluorescently conjugated extracellular mAbs. Intracellular staining for cytokines and transcription factors was performed using the Fixation/Permeabilization Solution Kit (eBiosciences). Useful human flow cytometry antibodies included CD45 (HI30), CD3 (UCHT1), CD4 (A161A1), CD8 (HIT8a), CD44 (BJ18), TNFα (MAb11), IFNγ (4S.B3), and Epcam (9C4); all Biolegend. Flow cytometry was carried out on the LSR-II flow cytometer (BD Biosciences). Data were analyzed using FlowJo v.10.1 (Treestar, Ashland, Oreg.).

### Example 3. Assessment of Galectin-9 Levels in Plasma and Serum of Cancer Patients

Plasma and serum Galectin-9 levels were assessed in patient samples and compared to healthy volunteers. Blood (10 ml) was drawn from peripheral venous access from 10 healthy controls and 10 inoperable cancer patients. Serum and plasma were extracted from each sample of blood. Blood was collected in standard EDTA tubes PicoKine™ ELISA; Catalog number: EK1113 was used essentially according to manufacturer's instructions. Results of individual values are tabulated in Table 2 and Table 3.

TABLE 2
Patient Samples

| | | Serum | Plasma |
|---|---|---|---|
| Cancer Type | Patient No. | (pg/ml) | (pg/ml) |
| Breast cancer with metastases in liver and bones | Patient 1 | 11362.29 | 12107.56 |
| Melanoma brain and lung metastases braf + | Patient 2 | 978.97 | 1106.79 |
| Melanoma lung metastases braf − | Patient 3 | 838.83 | 695.08 |
| Rectal cancer with liver metastases | Patient 4 | 579.42 | 725.62 |
| Locally advanced gastric cancer | Patient 5 | 666.67 | 645.2 |
| Gastric cancer with liver, spleen and adrenal | Patient 6 | 674.3 | 877.69 |
| metastases | | | |
| Stage III ovarian cancer | Patient 7 | 1439.61 | 1341.6 |
| Metastatic cancer of unknown primary | Patient 8 | 1432.39 | 1671.8 |
| Testicular cancer | Patient 9 | 1352.56 | 1696.11 |
| Sarcoma | Patient 10 | 968.18 | 1073.57 |
| | Average | 2029.322 | 2194.102 |

TABLE 3
Healthy Volunteer Samples

| Sample Number | Serum | Plasma |
|---|---|---|
| Control 1 | 536.4 | 611.97 |
| Control 2 | 476.43 | 592.58 |
| Control 3 | 612.66 | 651.43 |
| Control 4 | 269.75 | 414.41 |
| Control 5 | 460.26 | 602.28 |
| Control 6 | 206.66 | 405.8 |
| Control 7 | 385.88 | 439.85 |
| Control 8 | 525.283 | 654.2 |
| Control 9 | 711.047 | 718.68 |
| Control 10 | 296.85 | 349.09 |
| Average | 448.122 | 544.029 |

The above results show that high levels of Galectin-9 were detected in the plasma and serum samples of cancer patients (e.g., breast cancer patient, melanoma patient, ovarian cancer patient, testicular cancer patient, and sarcoma patient) as compared with healthy controls.

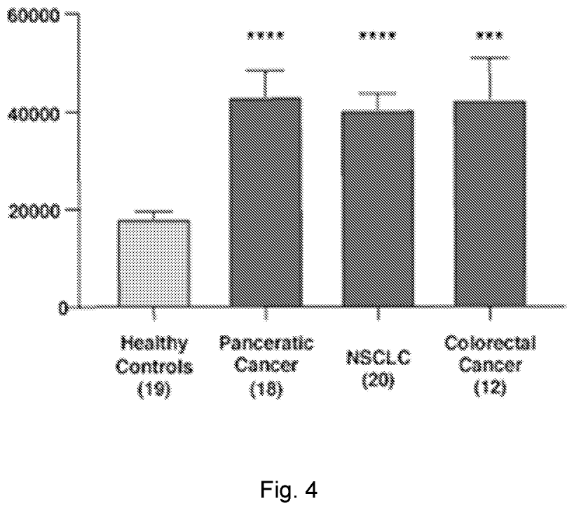

Follow on studies using the same procedures described above for serum measurements for in a larger number of healthy controls (19 controls) and subjects (18 subjects with pancreatic cancer, 20 subjects with non-small cell lung cancer (NSCLC), and 12 subjects with colorectal cancer) are shown in FIG. 4. Briefly, Galectin-9 levels in patient sera (sample size in parentheses) was measured using enzyme-linked immunosorbant assay (ELISA). The ELISA was run in sandwich format using the Human Galectin-9 PicoKine ELISA kit (Bolster Biological Technology). Patient serum galectin-9 levels were compared to healthy human serum controls. Statistical analysis performed by unpaired Student's t-test. (*p<0.05; **p<0.01; ***p<0.001; ****p<0.0001) Results show Galectin-9 levels in serum is significantly increased in cancer patients. Table 4 provides a values for the individual patient samples and Table 5 provides the average values.

TABLE 4
Blood Levels of Galectin-9 in Healthy Controls and Cancer Patients

| Healthy Controls | Panceratic Cancer | NSCLC | Colorectal Cancer |
|---|---|---|---|
| (Galectin-9 | (Galectin-9 | (Galectin-9 | (Galectin-9 |
| levels in pg/ml) | levels in pg/ml) | levels in pg/ml) | levels in pg/ml) |
| 22018 | 67781 | 63257 | 18146 |
| 15702 | 38064 | 19113 | 24952 |
| 13319 | 60664 | 21476 | 123670 |
| 13727 | 35114 | 45978 | 36430 |
| 15359 | 38212 | 51598 | 30573 |
| 14152 | 74782 | 24745 | 20868 |
| 10884 | 34932 | 40816 | 58977 |
| 15343 | 35196 | 37675 | 36385 |
| 8812 | 39558 | 40486 | 43970 |
| 17463 | 20916 | 78336 | 60134 |
| 27907 | 22917 | 48317 | 32573 |
| 15990 | 35654 | 30918 | 23227 |
| 14500 | 109239 | 25709 | |
| 32615 | 22223 | 42351 | |
| 21935 | 37523 | 22501 | |
| 18822 | 25310 | 42600 | |
| 20685 | 36620 | 43266 | |
| 25845 | 41730 | 41877 | |
| 19727 | | 33186 | |
| | | 54602 | |

TABLE 5
Average Blood Levels of Galectin-9 in
Cancer Patients Relative to Healthy Controls

| | Healthy | Pancreatic | | Colorectal |
|---|---|---|---|---|
| | Control | Cancer | NSCLC | Cancer |
| Average (pg/ml) | 18148 | 43135 | 40440 | 42492.08333 |
| Standard Deviation | 5968.8331 | 22139.526 | 14767.5495 | 28964.95828 |
| SEM | 1369.3442 | 5218.336262 | 3302.124452 | 8361.46323 |
| P-value | | 3.46372E−05 | | 0.001238666 |

Galectin-9 serum levels in patients with pancreatic cancer having various tumor burden are provided in Table 6 below:

TABLE 6
Correlation Between Galectin-9 Serum Levels and Tumor Burden

| Patient | Galectin-9 levels | |
|---|---|---|
| Number | serum (pg/ml) | Tumor burden |
| 1 | 19774 | low tumor burden, only locally advanced |
| 2 | 67781 | high tumor burden, multiple liver mets |
| 3 | 60664 | high tumor burden, multiple liver mets |
| 4 | 74782 | low tumor burden, only locally advanced |
| 5 | 20916 | low tumor burden, only locally advanced |
| 6 | 109239 | high tumor burden, multiple liver mets and |
| | | lymphnode mets |
| 7 | 22223 | low tumor burden, only locally advanced |

### Example 4. Assessment of Galectin-9 Expression and Localization using Immunohistochemical Analysis

The ability to use immunohistochemical analysis to determine Galectin-9 expression levels in tumors was assessed using paraffin-embedded biopsy-derived tumor samples.

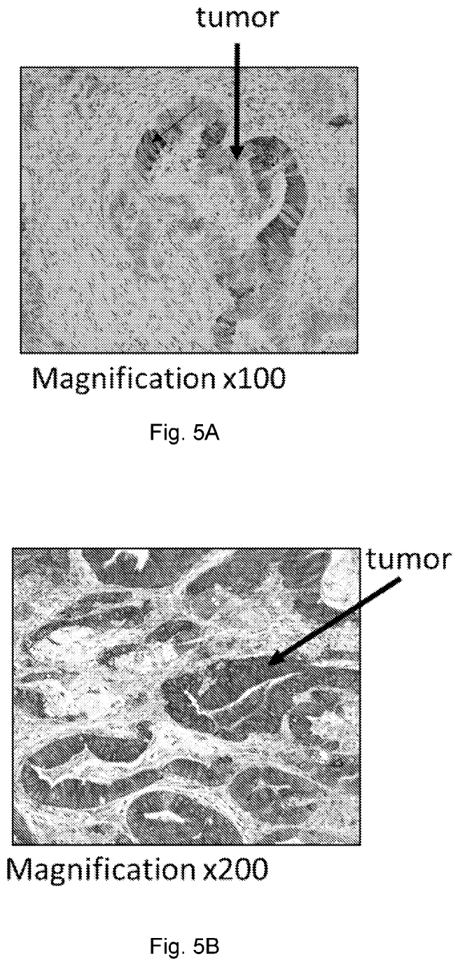

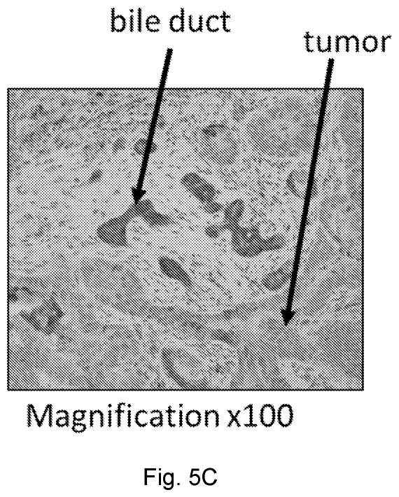

In brief, slides were deparaffinized (xylene: 2×3 min; absolute alcohol: 2×3 min., methanol: 1×3 min) and rinsed in cold tap water. For antigen retrieval, citrate buffer (pH 6) was preheated to 100 C in a water bath and slides were incubated in citrate buffer for 5 minutes. Slides were left to cool for about 10 min at room temperature and put in running water. Slides were washed in PBS, a pap pen circle was drawn around the section, and sections were incubated in blocking buffer (DAKO-Peroxidase blocking solution-S2023) for 5 minutes. Serum free blocker was added (Novocastra serum free Protein Blocker),and then rinsed off with PBS. Primary antibody (Sigma, anti-Galectin-9 clone 1G3) was used at 1:2000 dilution in DAKO-S2022 diluent and sections were incubated over night at 4C. Slides were washed with PBS and then incubated with the secondary antibody (anti- mouse) for 45 minutes at room temperature. Slides were washed and stained with ABC VECTOR STAIN (45 mins), washed with PBS, stained with DAB (1 ml stable DAB buffer+1 drop DAB)) for 5 minutes and washed in running water. Haematoxylin was added for 1 minute and 70% ETOH+1% HCL was applied to avoid over staining. Slides were left in running water for 2-3 min, then dipped in water, then absolute alcohol, and then xylene, 2 times for 30 seconds each. Cover slip and images were captured. Galectin-9 staining in a chemotherapy treated colorectal cancer and a liver metastasis of colorectal carcinoma are shown in FIGS. 5A and 5B. Results from Galectin-9 negative cholangiocarcinoma is shown in FIG. 5C.

### EQUIVALENTS

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

While several inventive embodiments have been described and illustrated herein, those of ordinary skill in the art are readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art are readily appreciate that all parameters, dimensions, materials, and configurations described herein are meant to be exemplary and that the actual parameters, dimensions, materials, and/or configurations are depend upon the specific application or applications for which the inventive teachings is/are used. Those skilled in the art are recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” are refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1.-60. (canceled)

61. A method for identifying a human cancer patient and treating the human cancer patient, the method comprising:
(i) providing a biological sample of a subject suspected of having a cancer,
(ii) measuring the level of galectin-9 in the biological sample,
(iii) identifying the subject as having the cancer or being at risk for the cancer based on the level of galectin-9 in the biological sample, wherein an elevated level of galectin-9 in the biological sample of the subject relative to a predetermined reference level indicates that the subject has the cancer or is at risk for the cancer and wherein the cancer is breast cancer, colorectal colon cancer (CRC), pancreatic cancer, or non-small cell lung cancer (NSCLC), and
(iv) administering to the subject an effective amount of an anti-Galectin-9 antibody, wherein the anti-Galectin-9 antibody comprises a light chain complementarity determining region 1 (CDR1) set forth as SEQ ID NO: 1, a light chain complementary determining region 2 (CDR2) set forth as SEQ ID NO: 2, and a light chain complementary determining region 3 (CDR3) set forth as SEQ ID NO: 3 and comprises a heavy chain complementarity determining region 1 (CDR1) set forth as SEQ ID NO: 4, a heavy chain complementary determining region 2 (CDR2) set forth as SEQ ID NO: 5, and a heavy chain complementary determining region 3 (CDR3) set forth as SEQ ID NO: 6.

62. The method of claim 61, wherein the biological sample is a blood sample.

63. The method of claim 62, wherein the blood sample is a serum sample or a plasma sample.

64. The method of claim 62, wherein the level of galectin-9 is measured by an immunoassay.

65. The method of claim 61, wherein the method further comprises determining tumor burden of the subject or cancer patient based on the level of galectin-9 in the biological sample.

66. The method of claim 61, wherein the method further comprises determining metastatic status of the cancer in the subject based on the level of galectin-9 in the biological sample.

67. The method of claim 61, wherein the cancer is pancreatic cancer.

68. The method of claim 67, wherein the pancreatic cancer is pancreatic ductal adenocarcinoma (PDA).

69. The method of claim 61, wherein the method further comprises: performing one or more additional diagnostic assays to confirm occurrence of the cancer or tumor burden of the cancer.

70. The method of claim 61, wherein the anti-Galectin-9 antibody comprises a light chain variable domain of SEQ ID NO: 8, and a heavy chain variable domain of SEQ ID NO: 7.

71. The method of claim 70, wherein the anti-Galectin-9 antibody is an IgG1 or IgG4 molecule.

72. The method of claim 71, wherein the anti-Galectin-9 antibody comprises a heavy chain comprising the amino acid sequence of SEQ ID NO:19 and a light chain comprising the amino acid sequence of SEQ ID NO:15.

73. The method of claim 61, further comprising administering to the subject an effective amount of an anti-cancer therapy comprising a checkpoint inhibitor.

74. The method of claim 73, wherein the checkpoint inhibitor is an anti-PD-1 antibody or an anti-PD-L1 antibody.

75. The method of claim 74, wherein the anti-PD1 antibody is selected from the group consisting of pembrolizumab, nivolumab, tislelizumab or cemiplimab.

76. The method of claim 74, wherein the anti-PD1 antibody is tislelizumab.

77. A method of treating a human subject having a solid tumor comprising:
(i) providing a biological sample of a subject having a cancer,
(ii) measuring the level of galectin-9 in the biological sample,
(iii) identifying the subject as having the cancer based on the level of galectin-9 in the biological sample, wherein an elevated level of galectin-9 in the biological sample of the subject relative to a predetermined reference level indicates that the subject has the cancer, and wherein the cancer is breast cancer, colorectal colon cancer (CRC), pancreatic cancer, or non-small cell lung cancer (NSCLC), and
(iv) administering to the subject a therapeutically effective amount an anti-Galectin-9 antibody, wherein the anti-Galectin-9 antibody comprises a light chain complementarity determining region 1 (CDR1) set forth as SEQ ID NO: 1, a light chain complementary determining region 2 (CDR2) set forth as SEQ ID NO: 2, and a light chain complementary determining region 3 (CDR3) set forth as SEQ ID NO: 3 and comprises a heavy chain complementarity determining region 1 (CDR1) set forth as SEQ ID NO: 4, a heavy chain complementary determining region 2 (CDR2) set forth as SEQ ID NO: 5, and a heavy chain complementary determining region 3 (CDR3) set forth as SEQ ID NO: 6.

78. The method of claim 77, wherein the biological sample is a blood sample.

79. The method of claim 78, wherein the blood sample is a serum sample or a plasma sample.

80. The method of claim 79, wherein the level of galectin-9 is measured by an immunoassay.

81. The method of claim 80, wherein the method further comprises determining tumor burden of the subject or cancer patient based on the level of galectin-9 in the biological sample.

82. The method of claim 77, wherein the method further comprises determining metastatic status of the cancer in the subject based on the level of galectin-9 in the biological sample.

83. The method of claim 77, wherein the cancer is pancreatic cancer.

84. The method of claim 83, wherein the pancreatic cancer is pancreatic ductal adenocarcinoma (PDA).

85. The method of claim 77, wherein the method further comprises: performing one or more additional diagnostic assays to confirm occurrence of the cancer or tumor burden of the cancer.

86. The method of claim 77, wherein the anti-Galectin-9 antibody comprises a light chain variable domain of SEQ ID NO: 8, and a heavy chain variable domain of SEQ ID NO: 7.

87. The method of claim 86, wherein the anti-Galectin-9 antibody is an IgG1 or IgG4 molecule.

88. The method of claim 87, wherein the anti-Galectin-9 antibody comprises a heavy chain comprising the amino acid sequence of SEQ ID NO:19 and a light chain comprising the amino acid sequence of SEQ ID NO:15.

89. The method of claim 77, further comprising administering to the subject an effective amount of an anti-cancer therapy comprising a checkpoint inhibitor.

90. The method of claim 89, wherein the checkpoint inhibitor is an anti-PD-1 antibody or an anti-PD-L1 antibody.

91. The method of claim 90, wherein the anti-PD1 antibody is selected from the group consisting of pembrolizumab, nivolumab, tislelizumab or cemiplimab.

92. The method of claim 91, wherein the anti-PD1 antibody is tislelizumab.
